(19) United States
(12) Patent Application Publication
(10) Pub. No.: US 2021/0290468 A1
(43) Pub. Date: Sep. 23, 2021

(54) COMBINED REHABILITATION SYSTEM FOR NEUROLOGICAL DISORDERS

(71) Applicant: Burke Neurological Institute, White Plains, NY (US)

(72) Inventor: Susan Wortman-Jutt, Scarsdale, NY (US)

(21) Appl. No.: 17/192,338

(22) Filed: Mar. 4, 2021

Related U.S. Application Data
(60) Provisional application No. 62/992,462, filed on Mar. 20, 2020.

(51) Int. Cl. A61H 1/02; G16H 40/67; G16H 20/30; G06F 3/01; G06F 3/14; G06F 3/0481; G09B 19/00; G09B 7/00

(52) U.S. Cl. CPC A61H 1/0274; A61H 2201/1659; G16H 20/30; G06F 3/011; G06F 3/14; G06F 3/0481; G09B 19/00; G09B 7/00; A61H 2201/5033; A61H 2201/5043; A61H 2201/5046; A61H 2201/501; A61H 2201/1207; A61H 2201/1652; A61H 2201/5048; G16H 40/67

(57) ABSTRACT

Systems and methods are provided for providing subjects experiencing multiple neurological impairments to synergistically improve their speech, language and upper limb impairments simultaneously in a controlled, measured, and customized manner.

### CROSS-REFERENCE TO RELATED APPLICATIONS

This application claims benefit of U.S. Provisional Application No. 62/992,462, filed Mar. 20, 2020, the contents of which are hereby incorporated by reference.

### FIELD OF THE INVENTION

The field of the invention generally relates to various technological improvements in systems, methods, and program products used in therapy to achieve neurological recovery or rehabilitation by directly targeting speech-language or cognitive and upper limb impairments simultaneously so as to achieve synergistic effects.

### BACKGROUND OF THE INVENTION

Throughout this application various publications are referred to by full citations. The disclosures of these publications, and all patents, patent application publications and books referred to herein, are hereby incorporated by reference in their entirety into the subject application to more fully describe the art to which the subject invention pertains.

Stroke, brain injury and other neurological disorders are a major source of disability throughout the United States, affecting millions of patients and caregivers, at a huge cost to the healthcare system. Proper treatment of such disabilities often requires rehabilitation services associated with motor deficiencies as well as speech and/or language deficiencies.

Unfortunately, existing computerized systems are not capable of addressing multiple modality deficiencies in a single session. Since many of the tasks that are used to target language rehabilitation deficiencies are redundant and bearing, the success of such treatments is limited, and can make it difficult for the patient and/or therapists to have sufficient time and/or energy to address other therapeutic needs, such as motor rehabilitation.

Technological challenges with the existing delivery of these services are significant, including the time it takes to provide these services, maintaining consistency of application and measurement of progress, overcoming patient fatigue and/or lack of interest in performing repetitive and non-engaging motor tasks or speech-language tasks, addressing other rehabilitation needs, to name a few.

What is needed is technological improvements in systems, methods, and program products for neurological rehabilitation that are maximally effective and efficient and which can reduce time demands on patients undergoing therapy, and therapy providers, can maximize use of their time and accelerate rehabilitation, can improve engagement, can track progress and/or otherwise improve patient outcome.

### SUMMARY OF THE INVENTION

The field of the invention generally relates to various technological improvements in systems, methods, and program products used in therapy to achieve neurological recovery or rehabilitation by directly targeting speech-language or cognitive and upper limb impairments simultaneously.

Currently there is no single technology that simultaneously addresses speech-language and motor skill rehabilitations in patients who exhibit multiple deficits in these areas. The present invention addresses this by, inter alia, cross-modality rehabilitative methods, systems and devices that harness the synergistic benefits of concurrent speech-language and motor skill therapies. This accelerates and/or enhances patient recoveries from the multiple deficits, and reduces both patient and therapist time involvement to reach therapy targets, and enables simultaneous verification and modulation of both speech-language and motor skill rehabilitation. The systems and methods provide neurological rehabilitation that is maximally effective and efficient and which can improve engagement, can track progress and/or otherwise improve patient outcome.

There is currently no defined neurological recovery or rehabilitation system that directly targets both speech-language, cognitive and upper limb impairments simultaneously. Many neurologically impaired patients recovering from stroke and brain injury, as well as individuals with developmental neurological issues, would benefit from a rehabilitation system that directly targets multiple impairments concurrently. This would increase efficiency for hospitals and rehabilitation centers and would increase the level of intensity of treatment for patients. The present invention addresses this and provides methods and systems to target multiple neurological impairments concurrently, and provide synergistically enhanced outcomes. This provides benefits to patients, and also to the benefits to the healthcare providers and therapists. The use of a combined platform to target speech-language, cognition and hand-arm movement deficits simultaneously enhances therapists' efficiency and reduces therapists' fatigue (by increasing the number of repetitions that can be performed without tiring the therapist). For hospitals and clinics, this means enhanced efficiency as well, and the potential to treat a larger number of patients, even in a group setting.

Examples of disorders in which multiple systems are affected include stroke, brain injury, neurological illness, Parkinson's disease and other degenerative disorders, as well as developmental disorders such as cerebral palsy and autism. The inventor is not aware of any video-based exercises being used with upper limb robotic systems that address speech, language and cognition; whereas, the invention herein permits patients experiencing multiple neurological impairments to work on both their speech, language and upper limb impairments simultaneously in a carefully controlled, measured, and customized manner. For example, tablet app or PC app-based speech-language therapies are configured to comprise a software system for an upper-limb rehabilitation robot, so that the robotic arm moves across the programmed vectors required for enhanced arm movement while aiming for images, letters and words that are custom-programmed by the patient's speech-language pathologist to address each patient's cognitive rehabilitation needs. Health providers who would benefit from a cross-modality rehabilitation device would include hospitals, rehabilitation centers, and the military, making it useful in multiple settings.

In embodiments, a method is provided of enhancing recovery from a non-fluent aphasia in a subject comprising:
a) obtaining from a therapy provider at least one language goal and at least one motor goal for a subject;
b) providing the subject with a visual display of one or more language tasks associated with the at least one language goal, wherein the visual display is operationally connected to a computer processor, and which language tasks are accomplished by an action comprising completing a motor task associated with the at least one motor goal, which motor task comprises movement along a predetermined path, from a predefined starting area to a predefined end area, of a moveable member of a robotic upper limb device operationally connected to a computer processor, which moveable member is moved by movement of an upper limb of the subject harnessed in at least a portion of the moveable member, and wherein movement of the moveable member by movement of the upper limb of the subject is translated into corresponding cursor movement on the visual display;
c) eliciting the subject to accomplish the one or more language tasks by an action comprising completing the motor task via upper limb movement which is translated into cursor movement on the visual display, within a predefined time period, wherein movement outside of the predetermined path does not complete the motor task; and
d) displaying on the visual display an indicator of the one or more language tasks having been accomplished subsequent to completion of the motor task within the predefined time period, or displaying on the visual display an indicator of one or more language tasks not having been accomplished subsequent to non-completion of the motor task within the predefined time period.

In embodiments, a system is provided for therapeutic treatment of a subject having a non-fluent aphasia and/or speech-language developmental motor disorder and/or a motor-speech disorder, the system comprising:

a. a memory device, wherein the memory device is operable to perform the following steps:
i. obtain, from a care provider associated with the subject via the system, at least one motor goal for the subject;
ii. obtain, from the care provider via the system, at least one language goal for the subject;
iii. store the at least at least one motor goal and the at least one language goal;
iv. obtain a plurality of treatments associated with a plurality of goals, wherein the plurality of treatments comprises:
   1. a plurality of motor tasks associated with a plurality of motor goals of the plurality of goals, the plurality of motor tasks comprising:
      a. one or more predefined paths;
      b. one or more predefined starting positions, each of the one or more predefined starting positions being associated with at least one of the one or more predefined paths; and
      c. one or more predefined finishing positions, each of the one or more predefined finishing positions being associated with at least one of the one or more predefined starting positions; and
   2. a plurality of language tasks associated with a plurality of language goals of the plurality of goals, wherein each of the plurality of language tasks is associated with at least one of the plurality of motor tasks such that each of the plurality of language tasks is at least partially accomplished by at least partially completing the at least one of the plurality of motor tasks; and
v. store the plurality of treatments;

b. one or more processor(s) operatively connected to the memory device, wherein the one or more processor(s) is operable to execute machine-readable instructions;

c. a robotic upper limb device comprising at least one movable member,

wherein the robotic upper limb device is operatively connected to the one or more processor(s) and

wherein the at least one movable member is operable to:
i. move along at least two axes; and
ii. mechanically couple with at least one upper limb of the subject;

d. an electronic device comprising input circuitry operatively connected to the one or more processor(s), wherein the electronic device is operable to:
i. receive one or more inputs from at least one of the following:
   1. the robotic upper limb device
   2. the subject; and
   3. the care provider; and
ii. communicate the one or more inputs from the electronic device to the one or more processor(s); and

e. a display operatively connected to the input circuitry, the robotic upper limb device, and the one or more processor(s), wherein the display is operable to provide one or more graphical user interfaces based on machine-readable instructions;

wherein the one or more processor(s) is operable to perform the following steps:
i. obtain, from the care provider associated with the subject via the input circuitry of the electronic device, one or more goals comprising:
   a. the at least one motor goal for the subject,
      wherein the at least one motor goal is associated with at least one motor task of the plurality of motor tasks; and
   b. the at least one language goal for the subject,
      wherein the at least one language goal is associated with at least one language task of the plurality of language tasks,
   wherein the one or more goals are stored in the memory operatively connected to the one or more processor(s);
ii. obtaining and executing first machine-readable instructions to display a first graphical user interface including a first visual display comprising:
   (A) a cursor indicating a relative position of the at least one movable member of the robotic upper limb,
   wherein the cursor is displayed at a predefined starting position at the beginning of the treatment;
   (B) one or more treatments associated with the one or more goals;
   (C) one or more prompts designed to elicit one or more actions by the subject directed towards accomplishing tasks associated with the one or more treatments; and
   (D) one or more indicators associated with the subject's progress of the one or more treatments,
   wherein the one or more indicators are updated in substantially real-time to reflect the subject's progress of the one or more treatments,
   wherein the execution of the first machine-readable instructions causes the display of the system to display the first graphical user interface,
   wherein the first visual display is operationally connected to the one or more processor(s) and the robotic upper limb device such that movement of the at least one movable member of the robotic upper limb device causes the cursor displayed on the display of the system to move in a manner reciprocal to the movement of the at least one movable member,
   wherein the one or more treatments comprise:
   (A) a first motor task of the at least one motor task,
   wherein the first motor task requires movement of the at least one movable member of the robotic upper limb device and the mechanically coupled at least one upper limb of the subject along a first predefined path of the plurality of predefined paths from a first predefined starting position of the plurality of predefined starting positions to a first predefined finishing position of the plurality of predefined finishing positions,
   wherein the first motor task is associated with the at least one motor goal for the subject; and
   (B) a first language task of the plurality of language tasks,
   wherein the first language task is at least partially accomplished by completing the first motor task;
iii. receiving, by the one or more processor(s), first data indicating movement of the at least one movable member,
   wherein the first data is stored in the memory operatively connected to the one or more processor(s);
iv. obtaining and executing second machine-readable instructions, causing the cursor displayed on the display of the system to move reciprocally with the movement indicated by the first data;
v. repeating steps (iii) and (iv) until the one or more processor(s) determine one or more of the following:
   a. the first motor task is completed;
   b. the first language task is completed; and
   c. a predetermined amount of time associated with the treatment has elapsed;
vi. obtaining and executing third machine-readable instructions to display a second graphical user interface including a second visual display comprising second data indicating one or more of the following:
   a. whether the first motor task was completed within the predetermined amount of time;
   b. whether the first language task was completed within the predetermined amount of time;
   c. a list of completed treatments; and
   d. a list of incomplete treatments;
wherein the execution of the third machine-readable instructions causes the display of the system to display the second graphical user interface, and
wherein the second data is stored in the memory operatively connected to the one or more processor(s).

In embodiments, also provided is a programmed product for therapeutic treatment of a subject having a non-fluent aphasia and/or speech-language developmental motor disorder and/or a motor-speech disorder, the system comprising:
1. a memory device, wherein the memory device is operable to perform the following steps:
   1. obtain, from a care provider associated with the subject via the system, at least one motor goal for the subject;
   2. obtain, from the care provider via the system, at least one language goal for the subject;
   3. store the at least at least one motor goal and the at least one language goal;
   4. obtain a plurality of treatments associated with a plurality of goals, wherein the plurality of treatments comprises:
      1. a plurality of motor tasks associated with a plurality of motor goals of the plurality of goals, the plurality of motor tasks comprising:
         1. one or more predefined paths;
         2. one or more predefined starting positions, each of the one or more predefined starting positions being associated with at least one of the one or more predefined paths; and
         3. one or more predefined finishing positions, each of the one or more predefined finishing positions being associated with at least one of the one or more predefined starting positions; and
      2. a plurality of language tasks associated with a plurality of language goals of the plurality of goals, wherein each of the plurality of language tasks is associated with at least one of the plurality of motor tasks such that each of the plurality of language tasks is at least partially accomplished by at least partially completing the at least one of the plurality of motor tasks; and
   5. store the plurality of treatments;
2. one or more processor(s) operatively connected to the memory device, wherein the one or more processor(s) is operable to execute machine-readable instructions;
3. configuration instructions to operate with a robotic upper limb device comprising at least one movable member,
   wherein the robotic upper limb device is operatively connected to the one or more processor(s) and
   wherein the at least one movable member is operable to:
   1. move along at least two axes; and
   2. mechanically couple with at least one upper limb of the subject;
4. an electronic device comprising input circuitry operatively connected to the one or more processor(s), wherein the electronic device is operable to:
   1. receive one or more inputs from at least one of the following:
      1. the robotic upper limb device
      2. the subject; and
      3. the care provider; and
   2. communicate the one or more inputs from the electronic device to the one or more processor(s); and
5. configures instructions to operate a display operatively connected to the input circuitry, the robotic upper limb device, and the one or more processor(s), wherein the display is operable to provide one or more graphical user interfaces based on machine-readable instructions;
   wherein the one or more processor(s) is operable to perform the following steps:
   i. obtain, from the care provider associated with the subject via the input circuitry of the electronic device, one or more goals comprising:
1. the at least one motor goal for the subject,
   wherein the at least one motor goal is associated with at least one motor task of the plurality of motor tasks; and
2. the at least one language goal for the subject,
   wherein the at least one language goal is associated with at least one language task of the plurality of language tasks,
   wherein the one or more goals are stored in the memory operatively connected to the one or more processor(s);
   ii. obtaining and executing first machine-readable instructions to display a first graphical user interface including a first visual display comprising:
   (A) a cursor indicating a relative position of the at least one movable member of the robotic upper limb,
   wherein the cursor is displayed at a predefined starting position at the beginning of the treatment;
   (B) one or more treatments associated with the one or more goals;
   (C) one or more prompts designed to elicit one or more actions by the subject directed towards accomplishing tasks associated with the one or more treatments; and
   (D) one or more indicators associated with the subject's progress of the one or more treatments,
   wherein the one or more indicators are updated in substantially real-time to reflect the subject's progress of the one or more treatments,
   wherein the execution of the first machine-readable instructions causes the display of the system to display the first graphical user interface,
   wherein the first visual display is operationally connected to the one or more processor(s) and the robotic upper limb device such that movement of the at least one movable member of the robotic upper limb device causes the cursor displayed on the display of the system to move in a manner reciprocal to the movement of the at least one movable member,
   wherein the one or more treatments comprise:
   (A) a first motor task of the at least one motor task,
   wherein the first motor task requires movement of the at least one movable member of the robotic upper limb device and the mechanically coupled at least one upper limb of the subject along a first predefined path of the plurality of predefined paths from a first predefined starting position of the plurality of predefined starting positions to a first predefined finishing position of the plurality of predefined finishing positions,
   wherein the first motor task is associated with the at least one motor goal for the subject; and
   (B) a first language task of the plurality of language tasks,
   wherein the first language task is at least partially accomplished by completing the first motor task;
   iii. receiving, by the one or more processor(s), first data indicating movement of the at least one movable member,
wherein the first data is stored in the memory operatively connected to the one or more processor(s);
   iv. obtaining and executing second machine-readable instructions, causing the cursor displayed on the display of the system to move reciprocally with the movement indicated by the first data;
   v. repeating steps (iii) and (iv) until the one or more processor(s) determine one or more of the following:
1. the first motor task is completed;
2. the first language task is completed; and
3. a predetermined amount of time associated with the treatment has elapsed;
   vi. obtaining and executing third machine-readable instructions to display a second graphical user interface including a second visual display comprising second data indicating one or more of the following:
1. whether the first motor task was completed within the predetermined amount of time;
2. whether the first language task was completed within the predetermined amount of time;
3. a list of completed treatments; and
4. a list of incomplete treatments;
   wherein the execution of the third machine-readable instructions causes the display of the system to display the second graphical user interface, and
   wherein the second data is stored in the memory operatively connected to the one or more processor(s).

### BRIEF DESCRIPTION OF THE DRAWINGS

The above and related objects, features, and advantages of the present invention, will be more fully understood by reference to the following detailed description of the exemplary embodiments of the present invention, when taken in conjunction with the following exemplary figures, wherein:

FIG. 1 is a block diagram of a system for combined rehabilitation for one or more neurological disorders in accordance with exemplary embodiments of the present invention. The system, in embodiments, includes a computer system 102, a display 104, and a robotic upper limb device 106, which, together, provides combined rehabilitation for one or more neurological disorders the of which the subject 108 has been diagnosed. As shown in FIG. 1, the computer system 102 may communicate with the display 104 and/or the robotic upper limb device 106 over network 50. Additionally, in embodiments, the display 104 and the robotic upper limb device 106 may communicate without the use of network 50.

FIGS. 1A-1 and 1B-1. 1A-1: In a prior art “Alphabetize Pictures” task for tablet (e.g., the Constant Therapy® tablet app, from Learning Corp., Newton, Mass., USA) a subject moves pictures with his/her finger to empty boxes, in alphabetical order, largely using one type of movement (e.g., a downward stroke). This task addresses verbal/analytical reasoning, and optionally the subject can say words aloud while performing task. 1B-1: In a related task for embodiments of the disclosure, pictures are spread across the field so that the contralateral arm of the subject is required to cross vectors when selecting the appropriate picture. To accomplish the language task in this example thus requires multiple upper limb movements as well as alphabetized ordering to get the correct answer. The predetermined path can start, for example, from an area encompassing the “pig”, but not the other pictured objects, on the visual display and end at one of the numbered boxes as a predefined end area. This enhances verbal/analytical reasoning while also targeting upper limb range-of-motion. Optionally, the subject can also be prompted to say words aloud while performing task in order to accomplish the task. In this manner, reciprocal areas of hand-arm movement and speech-language are engaged simultaneously in the cortex. This is consistent with how hand-arm and speech-language areas are engaged during normal activities of daily living, such as when one gestures when speaking.

FIGS. 1A-2 and 1B-2. 1A-2: In a prior art “Copy Words” task for tablet (e.g., the Constant Therapy® tablet app) the subject sees the word on the left and selects letters on the right to spell the word by dragging the appropriate letters across the screen with his/her finger. The selected letters appear in the empty boxes. This task addresses linguistic recall, phonological and morphological skills. Similarly, in a more advanced spelling task, “Spell What You See” task for tablet (e.g., the Constant Therapy® tablet app), a picture of the object appears, rather than the word itself 1B-2: In a related task for embodiments of the disclosure, the target word is centered and the letters appear around the word at wide vectors across the screen. The subject uses contralateral upper limb by way of the moveable member of the robotic upper limb device to select the correct letters by dragging them across the screen. The same template can be used for the more complex spelling task where the picture, rather than the word, placed in the center. These language tasks enhance target linguistic recall, phonological and morphological skills, along with upper limb range-of-motion. This is particularly useful for reinforcing connections between word structure and hand-arm movement that are used in written language, and also engaging pathways used in verbal word finding.

FIGS. 1A-3 and 1B-3. 1A-3: In a prior art “Identify Picture Categories” task for tablet (e.g., the Constant Therapy® tablet app) the subject sees the object on the left, and identifies the correct category from a field of 3 choices. The subject uses his/her finger to tap on the correct category. This addresses reading comprehension at a word-to-phrase level as well as word retrieval. 1B-3: In a related task for embodiments of the disclosure, the subject sees the object placed in the center of the screen, and identifies the correct category from a field of 3 choices that are spread across the screen at targeted vectors. The patient uses their contralateral upper limb movement, not simple finger tapping, to identify the correct category. This enhances reading comprehension at a word-to-phrase level as well as word retrieval and upper limb range-of-motion. Word retrieval and hand-arm movement are located in the left frontotemporal brain region, which would be simultaneously engaged during this task.

FIGS. 1A-4 and 1B-4. 1A-4: In a prior art “Name Verbs” task for tablet (e.g., the Constant Therapy® tablet app) the subject presses “start” with his/her finger and says the target verb, in this case “brushing,” into the microphone which inputs into the tablet. The software algorithm determines whether the word was said correctly. The Speech-Language pathologist may override the decision in cases where patients have more severe motor-speech disorders, so that reasonable verbal approximations are rewarded. This addresses action word retrieval. This is important as verbs are used to expand verbal utterances with greater frequency than nouns. For example, the word “brushing” can be used in many expandable contexts (e.g., brushing hair, brushing teeth, brushing a horse, brushing paint on a wall, “brushing someone off,” etc.) whereas the word “brush,” a noun, is not as readily expandable into multiple contexts. For this reason, verb retrieval is considered an important domain in aphasia therapy. A similar task, “Name Pictures,” tablet (e.g., the Constant Therapy® tablet app) for nouns, is also available and is constructed in the same manner. 1B-4: In a related task for embodiments of the disclosure, to accomplish the language task the subject is required to press “start” at multiple locations across several vectors on the predetermined path using the contralateral arm to move the moveable member, and says the verb (e.g., “brushing”) into a head-mounted microphone. The software algorithm of the system determines whether the word was said correctly each time. The user, such as a speech-language pathologist, may override the decision in cases where patients have more severe motor-speech disorders, so that reasonable verbal approximations are rewarded. The multiple repetitions of the word increases the intensity of the exercise and provides reinforcement. This exercise enhances action word retrieval as well as upper limb range-of motion. This is important since verb retrieval may be enhanced using upper limb movement. Further, upper limb strength and range-of-motion may be enhanced when naming action words. The dual use of upper limb movement and naming verbs engages reciprocal brain areas. The corresponding picture task, for nouns, works in a similar fashion.

FIGS. 1A-5 and 1B-5. 1A-5: In prior art task (Bionik InMotion™ Arm software) the subject attempts to get the ball in the center hole from each visualized point. This targets arm range-of-motion as well as strength and endurance of the upper limb. 1B-5: In a related task for embodiments of the disclosure, this “ball in the hole” can be reconfigured into a “scrambled word” exercise in which to accomplish the task the subject the subject spells a word by reaching for the correct letters through a series of predefined paths via upper limb directed movement of the moveable member of the robotic upper limb device, in order, at different vectors across the screen, in this case spelling “FROG.” Each time the correct letter is brought toward the center point, it appears below the circle, until the word is spelled. This enhances spelling and verbal word finding, as well as upper limb strength, endurance and range-of-motion.

FIG. 2A-1-2A-2: Illustration of a commercially available end-effector type robotic upper limb device (Armeo™). 2A-1: Showing subject sitting in position with injured arm harnessed in position on the device. 2A-2: Close-up of upper limb in place in a moveable member with optional hand open-close function portion.

FIG. 2B-1-2B-3: Illustration of a commercially available exoskeleton type robotic upper limb device (Tenoexo™). 2B-1: Showing exoskeleton type robotic upper limb device on arm with hand grasping ball. 2B-2: Showing exoskeleton type robotic upper limb device on arm from side view. 2B-3: Showing exoskeleton type robotic upper limb device on arm from top view.

FIG. 3A-3C: Illustration of upper limb movements. 3A: Supination (left side of image) and pronation (right side of image). From https://www.kenhub.com/en/library/anatomy/pronation-and-supination. 3B: Extension and flexion of elbow joint. 3C: Extension and flexion of wrist joint.

FIG. 3D-3I: Graphic showing flexion, extension, abduction, adduction, circumduction and rotation. 3D: flexion. 3E: extension. 3F: flexion and extension. 3G: flexion and extension. 3H: abduction, adduction, circumduction. 3I: rotation. (See BC Campus: Open Textbooks, Anatomy and Physiology, Chapter 9, Joints (59)9.5: Types of Body Movements. https://opentextbc.ca/).

FIG. 3J-3N: Upper limb movements from American Council on Exercise (2017). Muscles That Move the Arm; Ace Fitness: Exercise Science, on the worldwide web at acefitness.org/fitness-certifications/ace-answers/exam-preparation-blog/3535/muscles-that-move-the-arm/. 3J: Abduction and adduction. 3K: Flexion and extension. 3L: Internal and external rotation. 3M: Internal and external rotation. 3N: Horizontal abduction & horizontal adduction.

FIG. 3P-3T: Uniplanar, biplanar and multiplanar axis of rotation upper limb movements, from Edwards, Makeba (2017). Axis of Rotation; Ace Fitness: Exercise Science on the worldwide web at acefitness.org/fitness-certifications/ace-answers/exam-preparation-blog/3625/axis-of-rotation/. 3O: Uniplanar. 3P: Biplanar. 3Q: Biplanar. 3R: Multiplanar. 3S: Multiplanar. 3T: Multiplanar. (Humerus 302, ulna 304, phalanx 306, scapula 308).

FIG. 4 is a flowchart illustrating an exemplary process for combined rehabilitation for one or more neurological disorders in accordance with exemplary embodiments of the present invention.

FIG. 4A is another flowchart illustrating an exemplary process for combined rehabilitation for one or more neurological disorders in accordance with exemplary embodiments of the present invention.

### DETAILED DESCRIPTION OF THE INVENTION

The field of the invention generally relates to various technological improvements in systems, methods, and program products used in therapy to achieve neurological recovery or rehabilitation by directly targeting speech-language or cognitive and upper limb impairments simultaneously.

Embodiments of the present invention described herein avoids the prior art issues of separate patient time involvement, care provider time involvement, and coordinated recoveries in speech-language therapy and motor skill therapy. Embodiments of the present invention described herein maximize recovery times, efficient use of spatial and temporal resources, and provide synergistic outcomes in speech-language skill recovery and motor skill recovery.

In embodiments, a method is provided of enhancing recovery from a non-fluent aphasia in a subject comprising:
a) obtaining from a therapy provider at least one language goal and at least one motor goal for a subject;
b) providing the subject with a visual display of one or more language tasks associated with the at least one language goal, wherein the visual display is operationally connected to a computer processor, and which language tasks are accomplished by an action comprising completing a motor task associated with the at least one motor goal, which motor task comprises movement along a predetermined path, from a predefined starting area to a predefined end area, of a moveable member of a robotic upper limb device operationally connected to a computer processor, which moveable member is moved by movement of an upper limb of the subject harnessed in at least a portion of the moveable member, and wherein movement of the moveable member by movement of the upper limb of the subject is translated into corresponding cursor movement on the visual display;
c) eliciting the subject to accomplish the one or more language tasks by an action comprising completing the motor task via upper limb movement which is translated into cursor movement on the visual display, within a predefined time period, wherein movement outside of the predetermined path does not complete the motor task; and
d) displaying on the visual display an indicator of the one or more language tasks having been accomplished subsequent to completion of the motor task within the predefined time period, or displaying on the visual display an indicator of one or more language tasks not having been accomplished subsequent to non-completion of the motor task within the predefined time period.

In embodiments, accomplishing the one or more language tasks comprises completion of movement along the predetermined path and subsequent selection by the subject of a predefined area of the visual display corresponding to a correct solution for the language task via a selection portion of the robotic upper limb device which is activatable by the subject so as to select an area of the visual display via the cursor on the visual display.

In embodiments, the selection by the subject of a predefined area of the visual display corresponding to a correct solution for the language task cannot be effected by the subject touching the screen of the visual display, nor by moving a touchpad-based cursor or mouse-based cursor which is not operationally connected to the robotic upper limb device.

In embodiments, the predefined area of the visual display corresponding to a correct solution for the language task is not the predefined starting area.

In embodiments, movement of the moveable member of the robotic upper limb device is adjustable by a non-subject user, or by software executed by the computer processor operationally connected thereto, so as to assist, perturb, constrain or resist motion of the moveable member of the robotic upper limb device by the upper limb movement of the subject.

In embodiments, the methods comprise eliciting the subject who has either failed to accomplish the language task after steps a), b), c) and d) have been performed or who has accomplished the language task after steps a), b), c) and d) have been performed, to accomplish a second or subsequent one or more language tasks by a second or subsequent iteration of steps c) and d).

In embodiments, the methods further comprise iteratively repeating a plurality of sets of steps c) and d), with a predetermined time period of non-performance in between each set of steps c) and d), so as to thereby enhance recovery in a subject from a non-fluent aphasia over a period of time or so as to thereby enhance speech-language therapy in a subject with a speech-language developmental motor disorder over a period of time.

In embodiments, movement resistance of the moveable member of the robotic upper limb device is adjusted or adjustable by software executed by the computer processor operationally connected thereto, so as to assist, perturb, constrain or resist motion of the moveable member of the robotic upper limb device by the upper limb movement of the subject.

In embodiments, movement resistance of the moveable member of the robotic upper limb device is adjusted in between one or more iterations of sets of steps a), b) and c) or one or more iterations of sets of steps b) and c).

In embodiments, adjustment is effected by a non-subject user, or by software executed by the computer processor operationally connected thereto, so as to assist, perturb, constrain or resist motion of the moveable member of the robotic upper limb device by the upper limb movement of the subject is proportional to accuracy of movement of the moveable member along the predefined path.

In embodiments, adjustment by a non-subject user, or by software executed by the computer processor operationally connected thereto, after a first set of steps a), b), c) and d) or a set of steps c) and d) is to assist motion of the moveable member of the robotic upper limb device by the upper limb movement of the subject wherein the subject's upper limb movement resulted in display on the visual display an indicator of language task non-completion.

In embodiments, adjustment by a non-subject user, or by software executed by the computer processor operationally connected thereto, after a first set of steps a), b), c) and d) is to resist motion of the moveable member of the robotic upper limb device by the upper limb movement of the subject wherein the subject's upper limb movement resulted in display on the visual display an indicator of language task completion.

In embodiments, at least one of the one or more language tasks is accomplished by an action comprising completing a motor task, which motor task comprises a plurality of individual movements, each along its own predetermined path from a predefined starting area to a predefined end area.

Language tasks may include, without limitation, speech production tasks, naming tasks, reading tasks, writing tasks, semantic processing tasks, sentence planning tasks, and/or auditory processing tasks. Language tasks involving verbalization may include, without limitation, syllable imitation, word imitation, and/or word repetition tasks.

Naming tasks may include, without limitation, rhyme judgment, syllable identification, phoneme identification, category matching, feature matching, picture naming (with or without feedback), and/or picture word inference tasks.

Reading tasks may include, without limitation, lexical decision, word identification, blending consonants, spoken-written word matching, word reading to picture, category matching, irregular word reading, reading passages, long reading comprehension, sound-letter matching, and/or letter to sound matching tasks.

Writing tasks may include, without limitation, word copy, word copy completion, word spelling, word spelling completion, picture spelling, picture spelling completion, word dictation, sentence dictation, word amalgamation, and/or list creation tasks.

Semantic processing tasks may include, without limitation, category identification, semantic odd one out, semantic minimal pairs, and/or feature matching tasks.

Sentence planning tasks may include, without limitation, verb/thematic role assignment, grammaticality judgment, active sentence completion, passive sentence completion, and/or voicemails tasks.

Auditory processing tasks may include, without limitation, spoken word comprehension, auditory commands, spoken sound identification, environmental sounds identification (picture or word), syllable identification, auditory rhyming, and/or phoneme to word matching tasks.

In embodiments, the language task comprises a verbal/analytical reasoning language task.

In embodiments, the language task comprises a linguistic recall, phonological and/or speech skill task.

In embodiments of the systems or methods, the language task comprises a cognitive skill task.

In embodiments, the method enhances a connection between word structure and hand-arm movement used in written language.

In embodiments, the method engages a pathway used in verbal word finding.

In embodiments, the language task comprises word identification of a pictured object category.

In embodiments, the method enhances reading comprehension at a word-to-phrase level and/or enhances word retrieval.

In embodiments, enhancement, relative to conventional speech-language therapy or to speech-language therapy not involving a concurrent or simultaneous movement of a subject's upper limb along a predefined path, is in a quantitative speech, language or cognitive outcome. For example, a subject treated by the method can experience enhanced recovery from non-fluent aphasia as compared to a comparable single modality therapy on a device and system method as descried in U.S. Pat. No. 10,283,006, Anantha et al., issued May 7, 2019, hereby incorporated by reference in its entirety. In non-limiting examples, such include increasing the number of syllables, number of words, or number of sentences achieved by a subject within, for example, a set time period. In non-limiting examples, such include increasing the rate of recovery from a starting point in number of syllables, number of words, or number of sentences achieved by a subject within, for example, a set time period. In non-limiting examples, such include increasing the density or richness of syllables, words, or sentences achieved by a subject within, for example, a set time period. In non-limiting examples, such include increasing the rate of accomplishment, or absolute accomplishment amount, of language tasks by a subject within, for example, a set time period. For example, a treated subject can master tasks in half the time or less, or two thirds the time or less versus conventional therapy which does not combine the two modalities into a single therapy.

In embodiments, enhancement, relative to conventional motor therapy or to motor therapy not involving a concurrent or simultaneous performance of a language task, is in a quantitative motor outcome. In non-limiting examples, such include increasing the rate of accomplishment, or absolute accomplishment amount, of motor tasks by a subject within, for example, a set time period. For example, a treated subject can master tasks with significantly improved Fugl-Meyer scores and/or improved time on the Wolf Motor Function Test as compared with usual care or versus conventional therapy which does not combine the two modalities into a single therapy.

In embodiments, a left frontotemporal brain region in the subject is simultaneously engaged when accomplishing the one or more language tasks by completion of the motor task of movement.

In embodiments, the language tasks are only accomplished if, in addition to the action comprising completing a motor task which comprises movement along a predetermined path, the subject also verbalizes one or more words into a microphone device simultaneously or contemporaneously with the movement or completion of the movement. In embodiments, the microphone device is a head-mounted microphone device on the subject. In embodiments, the microphone device inputs into a computer processor. In embodiments, algorithm-based software determines whether the word said was sufficiently correct to accomplish the language task. In embodiments, a parameter of the algorithm-based software is user-adjustable such that a verbal approximation of a correct word is sufficient to accomplish the language task.

In embodiments, a language task comprises verb naming of an action illustrated on the visual display. In embodiments, the language task comprises noun naming of an object illustrated on the visual display.

In embodiments, multiple repetitions of the word and completion of the movement are required to accomplish the language task.

In embodiments, the method enhances word retrieval.

In embodiments, the language task comprises a spelling task requiring completion of multiple movements to accomplish the language task.

In embodiments, a word to be spelled for a language task comprises multiple letters and each letter requires completion of movement along a different predetermined path within the predefined time.

In embodiments, the language tasks on the visual display are presented in the form of a game, and wherein the gameplay comprises accomplishing the language tasks.

In embodiments, the user targets a speech and/or language goal for the subject and adjusts the language task(s) and/or motor task(s) in accordance with the speech and/or language goal for the subject, and/or in accordance with a motor goal for the subject.

In embodiments, the method comprises, or the system can receive from a user, a user-defined language goal and/or a motor goal for a subject. In embodiments, the system can receive from a user language goal and/or motor goal selection criteria for a subject. In embodiments, the criteria can be individual language goal and/or motor goal criteria. In embodiments, the criteria can be combined or dual language goal and motor goal criteria. The system may use the user-specified language goal and/or motor goal selection criteria to select language tasks for the subject. In response to the subject accomplishing and/or not accomplishing the tasks, the system may determine whether the subject's performance complies with specified criteria. In cases where the subject's performance does not comply with the criteria, the system may select a new language task for the user. In some cases, for example in an automated mode, the new task may be inconsistent with the user-specified task selection criteria. Thus, in embodiments, the system may override the user-specified task selection criteria based on the subject's performance.

Motor tasks can include movements that require at least a portion of a subject upper limb to move in a manner involving flexion, extension, pronation, supination, abduction, adduction, circumduction, rotation. The movement may be uniplanar, biplanar or multiplanar. The movement may involve one or more portions of the upper limb. Hand, wrist, forearm, elbow, upper arm and/or shoulder movement may be required. Shoulder joint, elbow joint and/or wrist joint movement may be required.

Predetermined paths, which can be user-defined or system-provided, may be selected or provided in order to engage one or more of flexion, extension, pronation, supination, abduction, adduction, circumduction, and/or rotation of the upper limb.

Motor tasks can be selected by the user or provided by system which are relevant to achieving the motor goal.

In embodiments, the system may use the user-specified language goal and/or motor goal selection criteria to select motor tasks for the subject. In response to the subject accomplishing and/or not accomplishing the tasks, the system may determine whether the subject's performance complies with specified criteria. In cases where the subject's performance does not comply with the criteria, the system may select a new motor task for the user. In some cases, for example in an automated mode, the new task may be inconsistent with the user-specified task selection criteria. Thus, in embodiments, the system may override the user-specified task selection criteria based on the subject's performance. The system and method may be personalized, e.g., by the user, to provide language tasks specific to a language goal and/or a motor goal selected by the user for the subject. The system may prompt or elicit the subject to perform one or more of the specific language tasks (which may involve one or more of language, speech, spoken and cognitive tasks). In response to a task prompt, the subject may perform the prompted language task.

Based on the completion of the task by the subject subsequent to the prompt, the system may determine whether the user has accomplished or not accomplished the task correctly. If the subject has not correctly accomplished the task, the system may prompt the subject to perform the task again. In embodiments, if the subject fails to accomplish the task one or more times, the system may prompt the subject to perform a different task. If the subject has correctly accomplished the task, the system may prompt the subject to perform a new task.

In embodiments, based on task accomplishment and/or non-accomplishment by the subject, the system may generate performance data characterizing the subject's performance.

In embodiments, the method is for enhancing recovery from a non-fluent aphasia in a subject. In embodiments the non-fluent aphasia is associated with a prior stroke or traumatic brain injury in the subject.

In embodiments, the subject has suffered a prior stroke.

In embodiments, the subject has suffered a prior traumatic brain injury.

In embodiments, the method is for enhancing speech-language therapy in a subject with a speech-language developmental motor disorder.

In embodiments, the speech-language developmental motor disorder is cerebral palsy.

In embodiments, the speech-language developmental motor disorder is a childhood developmental disorder.

In embodiments, the speech-language developmental motor disorder is associated with hemiplegic cerebral palsy, Angelman syndrome, fragile x syndrome, Joubert syndrome, terminal 22q deletion syndrome, Rett syndrome, or autism with motor difficulties.

In embodiments, the subject's oral-motor control is enhanced.

In embodiments, the robotic upper limb device is an end-effector type robotic upper limb device.

In embodiments, the robotic upper limb device is an exoskeleton type robotic upper limb device.

In an embodiment the subject is younger than 18 years old.

In an embodiment the subject is 18 years or older.

In embodiments the user is administering a language rehabilitative therapy and/or motor rehabilitative therapy to the subject. In embodiments, the user is a speech-language therapist or speech-language pathologist. In embodiments, the user is a clinician. In embodiments, the user is a care provider. A care provider may be any of a speech-language therapist, speech-language pathologist, and clinician.

In embodiments, the method enhances certain quantifiable speech-language therapy outcomes synergistically. In embodiments, the method enhances certain speech-language therapy outcomes synergistically and others concurrently. In embodiments, the method preferentially enhances the speech-language therapy outcomes improved synergistically as compared to those only improved concurrently. In embodiments, recovery is enhanced relative to the recovery seen or obtained from the same language tasks accomplishment but with no robotic arm motor requirement, e.g. wherein the tasks can be accomplished using, e.g., a touch screen control or a hand-controlled mouse not requiring any limb movement, only hand and finger movement.

In embodiments, the methods further comprise an initial step of providing a system which comprises a visual display operationally connected to a computer processor which executes software for one or more language tasks displayed on the visual display, and comprises a moveable member of a robotic upper limb device operationally connected to the computer processor which also executes software which tracks and can control movement of the moveable member of a robotic upper limb device and which system is configured to translate movement of the moveable member into corresponding cursor movement on the visual display. In embodiments the software can be an application. In embodiments, the software performs one or more operations associated with a motor goal and a language goal. In embodiments, software performs one or more operations associated with dual goals of a motor goal and a language goal.

Language goals are commonly determined in the art by speech-language therapists, physicians and other speech-language therapy providers. Motor goals are commonly determined in the art by speech-motor therapists, physicians and other motor therapy providers. Language goals may set individually for a subject, or set to standardized quantifiable speech-language therapy outcomes (e.g., as known in the art and as also discussed in this specification). Language goals can comprise any language domain, for example speech and/or related cognition. Motor goals may set individually for a subject, or set to standardized quantifiable motor therapy outcomes (e.g., as known in the art and as also discussed in this specification).

Rehabilitation robots can be programmed such that they reduce their level of support when patients begin to initiate movement independently, thereby retraining function. Additionally, they provide hundreds of repetitions for the patient, which a human occupational or physical therapist would otherwise not be able to provide. This can improve outcomes for the patients as compared to non-robot therapy, and can also reduce the burden on physical and occupational therapists and enhance efficiency for healthcare institutions. Herein, these advantages are synergistically effected by simultaneous combined rehabilitation of both language, cognitive and motor domains into a dynamic and robust form of neurological rehabilitation.

Robotic upper limb devices usable in the invention include exoskeleton type and end-effector type. (See Lee, S. H., Park, G., Cho, D. Y. et al. Comparisons between end-effector and exoskeleton rehabilitation robots regarding upper extremity function among chronic stroke patients with moderate-to-severe upper limb impairment. Sci Rep 10, 1806 (2020). End-effector type are connected to patients at one distal point, and their joints do not match with human joints. Force generated at the distal interface changes the positions of other joints simultaneously, making isolated movement of a single joint difficult. The device can provide sufficient and controllable end-effector forces for functional resistance training. If necessary, these can be applied in any direction of motion. The devices are capable of providing adjustable resistances based on subjects' ability levels. Exoskeleton type resemble human limbs as they are connected to patients at multiple points and their joint axes match with human joint axes. Training of specific muscles by controlling joint movements at calculated torques is possible.

Examples of commercial robotic upper limb devices for rehabilitation include Tenoexo™ (an exoskeleton type), Bionik™ (InMotion 2.0, Interactive Motion Technologies, Watertown, Mass., USA) (an end-effector type), ArmeoSpring™, ArmeoSenso™ and ArmeoPower™ (Hocoma, Switzerland), the PaRRo robot arm, the Pacifio robotic arm (Barrett Technology, Newton, Mass., USA), and the Yeecon robotic arm (Yeecon Medical Equipment Co., China), See also, for example, U.S. Pat. No. 7,618,381, issued Nov. 17, 2009, Krebs et al., hereby incorporated by reference in its entirety.

In embodiments, the robotic upper limb device comprises a dynamic robotic rehabilitation apparatus. In embodiments, the apparatus provides appropriate, and/or user-controllable, dynamic and sensory inputs to upper limb muscle groups occurring during normal upper arm movement (for example, grasping, reaching, lifting). In embodiments the predetermined path can emulate one or more of grasping, reaching, following, tracing, or lifting upper arm movements. In embodiments, a computer or apparatus associated with, or part of, the robotic upper limb device can effect actuation of one or more motors associated with a dynamic portion of the device to provide at least one of assistance, perturbation, and resistance to motion by the subject of the robotic upper limb device, including movement along a predetermined path. In embodiments, the robotic upper limb device comprises a moveable member which has a wrist attachment and/or forearm attachment and/or forearm support. In embodiments, the subject's upper limb is placed in a harness or attachment of the moveable member of the robotic upper limb device. In embodiments, the upper limb is constrained therein, e.g. by straps or the like, and movement by the subject of their upper limb thereby causes the moveable member of the robotic upper limb device. By “harnessed” in at least a portion of the moveable member as used herein, any form of attachment or touching of the upper limb to the moveable member which can effect movement of the member by movement of the upper limb is encompassed. Non-limiting examples include a subject's upper limb may be strapped in (e.g., by fabric velcro-type straps), clamped in by hard material (e.g., plastic constraints) or merely firmly inserted into an ergonomically shaped receiving portion of the member, or a portion gripped by the hand of the upper limb of the subject.

In embodiments, the movement of moveable member of the robotic upper limb device is controllable by the software in order to provide functional resistance training. Resistance to movement or assistance to movement parameters can be set by a user or by the software based on one or more algorithms, for example based on one or more prior attempts at movement of the moveable member by the subject. Functional resistance training is known in the motor rehabilitative art. As used herein, to resist motion does not mean to prevent motion absolutely, rather it means to provide resistance to motion which resistance can still be overcome by sufficient human upper limb muscle operation. Similarly, assistance (or reduced resistance relative to a previous resistance level) can be applied to the moveable member of the robotic upper limb.

In embodiments, the language task is completed simultaneously with the completion of movement along the predetermined path of the moveable member of the robotic upper limb device by the upper limb movement of the subject, or wherein the language task is completed simultaneously with selection by mechanical movement of a finger, hand or arm, of a predefined area of the visual display upon or subsequent to completion of movement along the predetermined path of the robotic upper limb device by the upper limb movement of the subject.

In embodiments, movement along the predetermined path of the robotic upper limb device operationally is processed as completed only if the movement is within predetermined spatial tolerance limits of movement. In embodiments, a user, such as a rehabilitative therapist or clinician, can select the spatial tolerance limits for the predetermined path prior to step a), prior to step b), or prior to step c). In embodiments, the software can select the spatial tolerance limits for the predetermined path prior to step a), prior to step b), or prior to step c), and can adjust them up or down based on quantification of a prior performance by the subject of the motor task. In embodiments, the spatial tolerance limits are 2D limits. In embodiments, the spatial tolerance limits are 3D limits.

In embodiments, the predetermined path comprises an arc, a straight line, a zigzag, or a serpentine shape. In embodiments, the predetermined path is a 2D vector. In embodiments, the predetermined path is a 3D vector. In embodiments, the predetermined path comprises one or more targeted vectors.

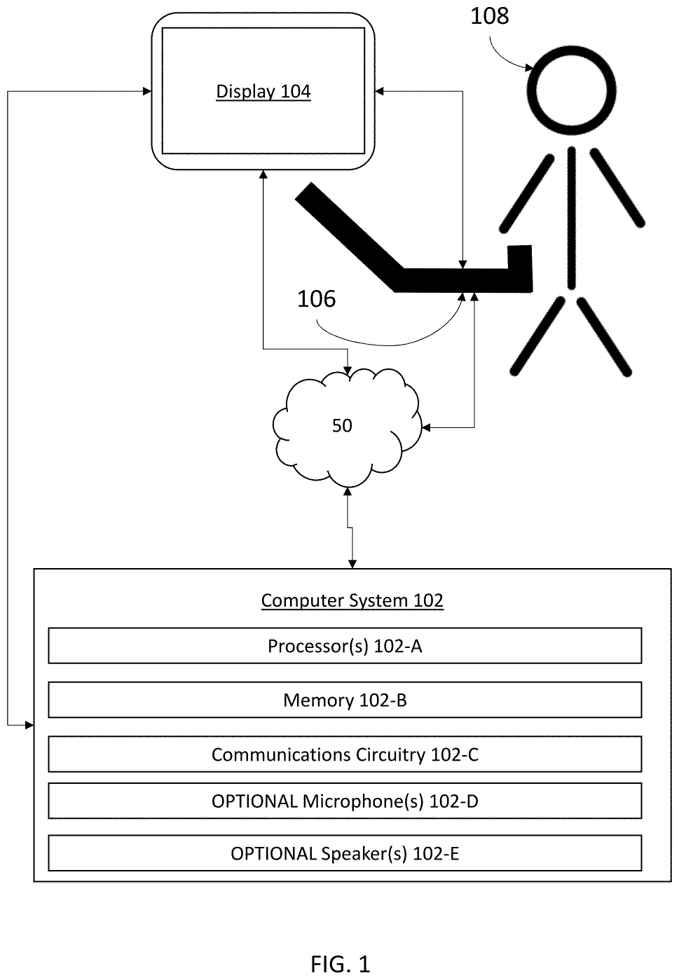

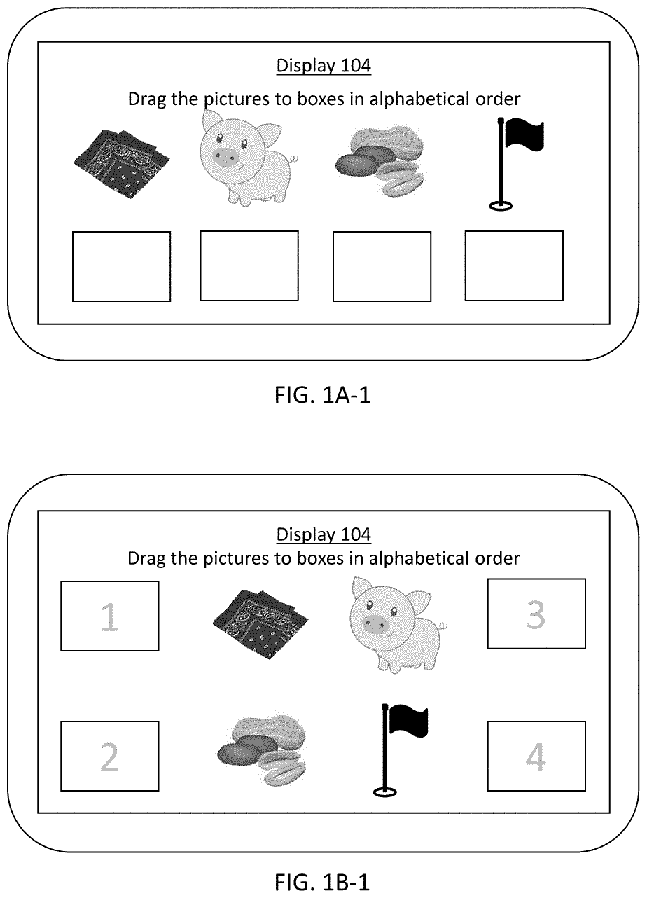

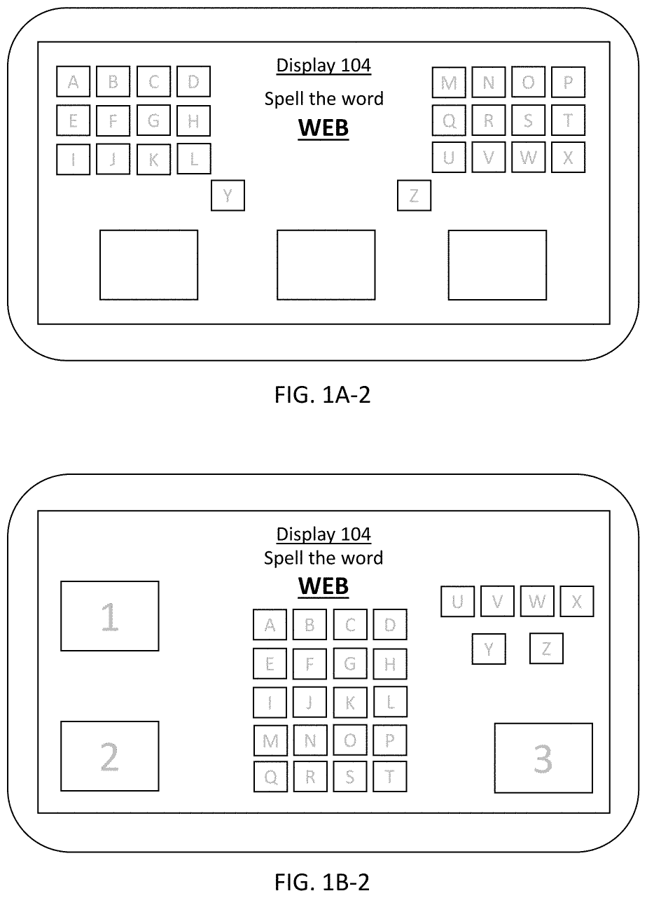

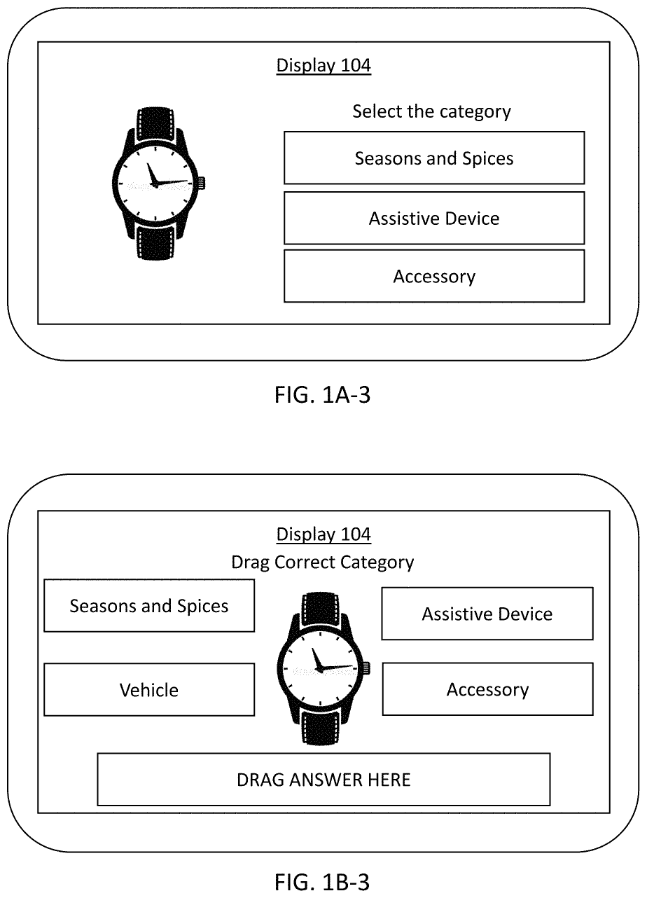

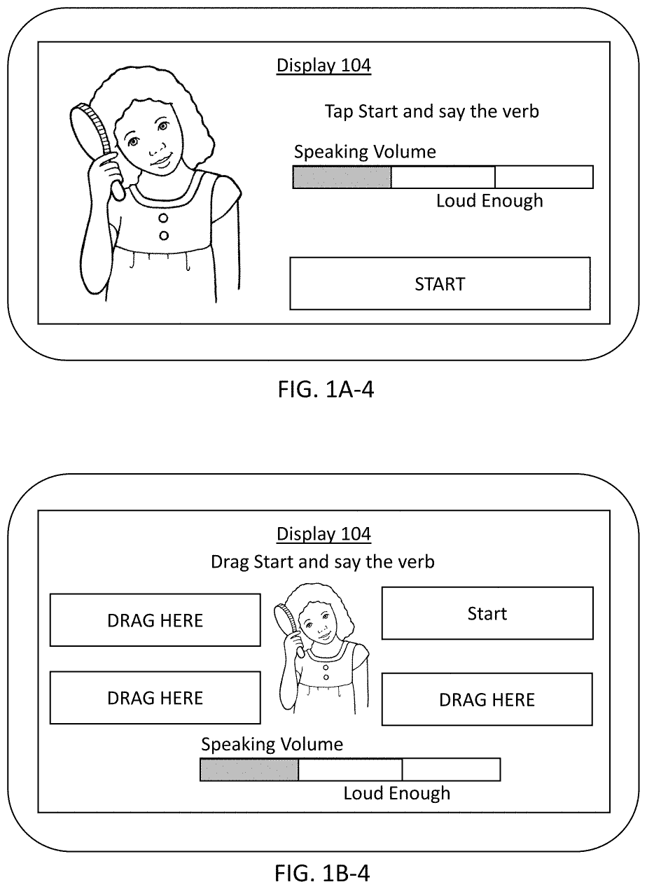

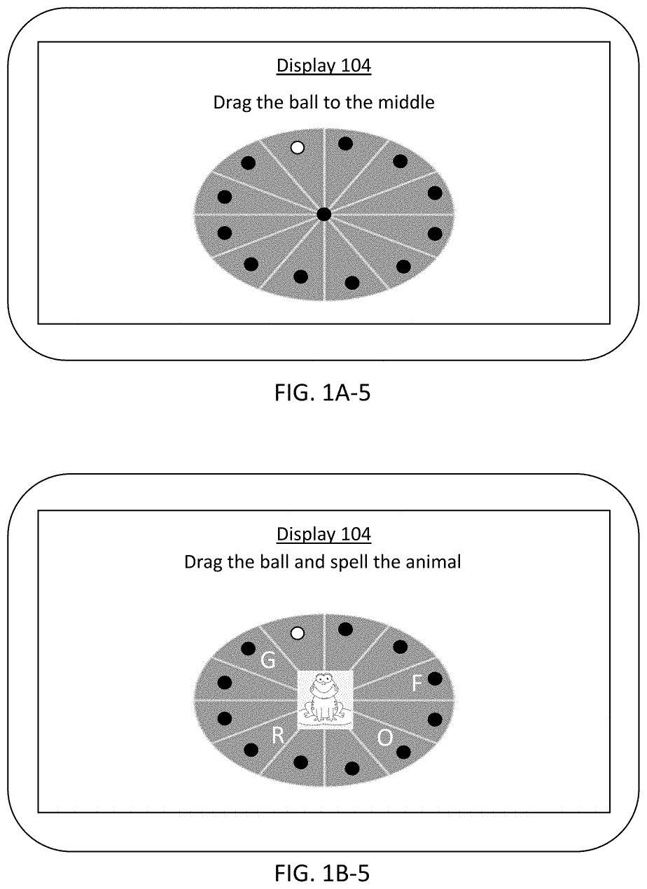

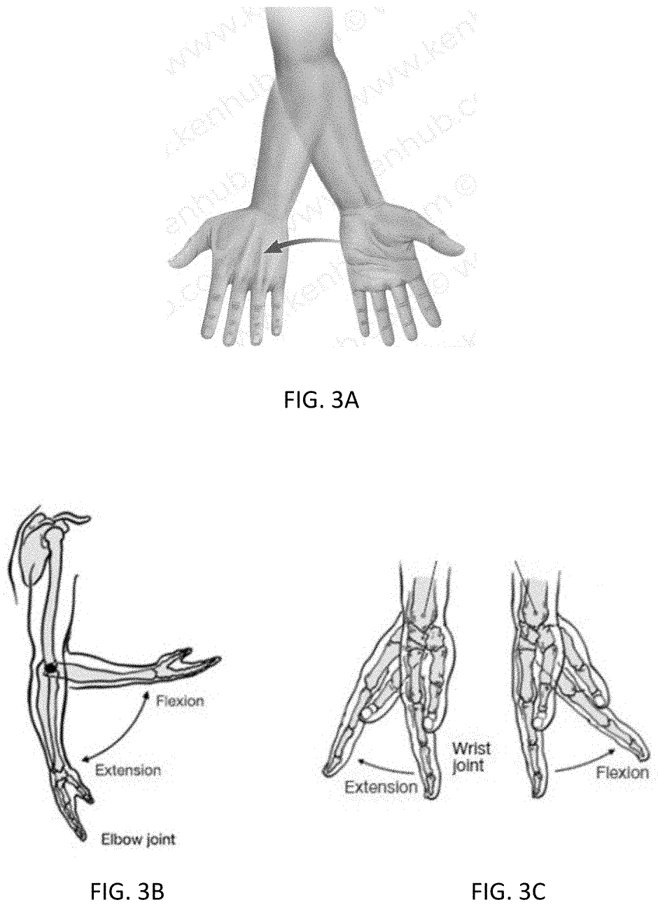

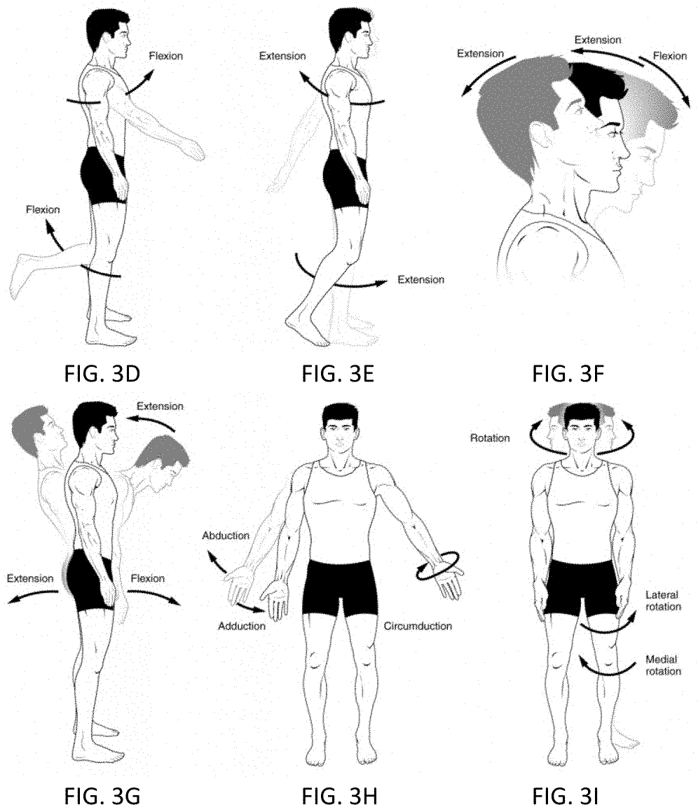

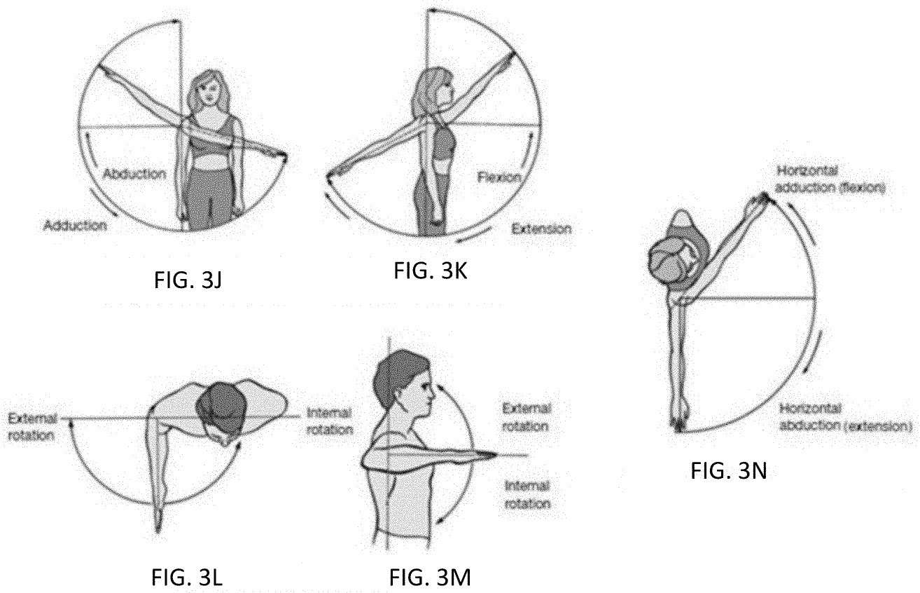

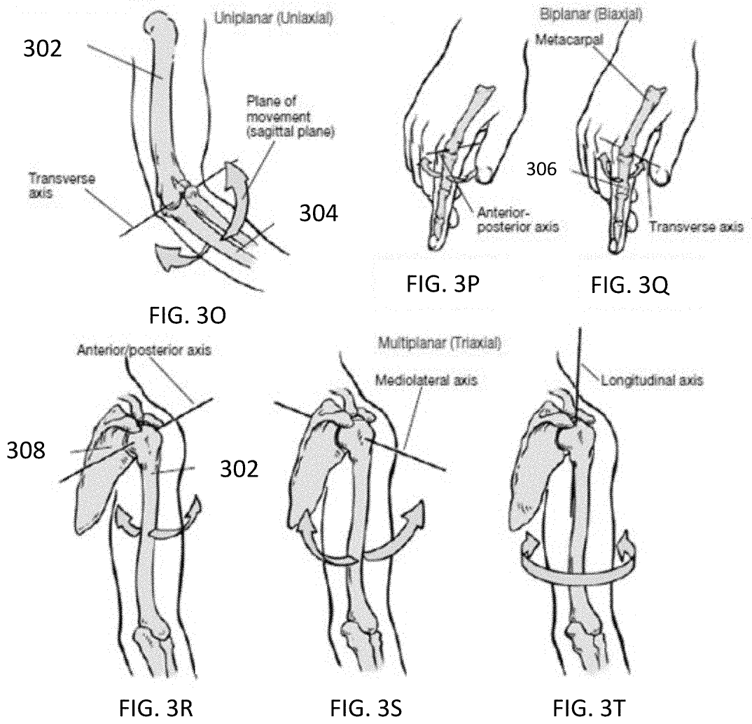

Any predefined time periods can be set by the user and/or implemented by the software. Any predetermined paths can be set by the user and/or implemented by the software in relation to the language task as appropriate. For example, in FIG. 1B-3, the path can be a vector across the screen to drag the correct category from a predefined starting area to the answer box as a predefined end area (as opposed to simply “clicking” on the correct category in prior art FIG. 1A-3). For example, in FIG. 1B-5, the paths can be a series of individual vectors across the screen to drag each correct letter, in order, to spell FROG correctly from each letter's predefined starting area (example shown in 1B-5) to the central answer box as a predefined end (as opposed to simply performing a motor task and no language task by dragging the ball-shaped virtual object to the center in prior art FIG. 1A-5). The predetermined paths may be set up so as to require movement of letters, words and/or images across the screen via movement of the moveable member from the subject's upper limb movement. Herein, instead of a patient moving letters, words or images across the screen with his or her finger to accomplish a language task, the patient (for example, with multiple neurological and motor deficits) uses movement by their upper limb of a moveable member of a robotic upper limb device to respond, via a corresponding cursor on the visual display (which cursor can take any form, e.g. cross hairs, geometric shape, dot, circle, image, etc.) to answer cognitively challenging questions (language tasks) that target their specific disability/disabilities.

In a non-limiting example, instead of in the field of choices for choosing a correct description of a presented image on a visual display, e.g., with the answer options “sweaters,” “carrots” and “the boys,” conventionally the answers are presented horizontally across a tablet screen visual display for ease of manual manipulation. The subject can “click” on the answer using a screen touch with their fingertip. However, by arranging predetermined paths that require, for example, the answer options to be placed along the trajectory of the robotic upper limb device moveable member movements, to answer questions correctly the subject must move the arm across the predetermined path (a prescribed trajectory for example). When performed to answer the correct answer this recruits related motor and language/speech/cognitive neurological paths which can interact and provide synergistic benefits in recovery not seen when the language and motor tasks are simply performed separately or sequentially. Additionally, for speech-language exercises requiring patients to verbalize their answers, a head-mounted microphone may be worn to engage verbally with the screen while the subject simultaneously moves, for example, an injured arm. Thus, subjects with speech-language, cognitive and/or motor deficits can advantageously have their speech-language, cognitive and/or motor deficit recoveries accelerated and/or enhanced relative to individual therapies.

In embodiments, a trigger operationally attached to the robotic upper limb device may be triggered by the hand or finger once the subject has completed movement along the predetermined path of the robotic upper limb device and an associated cursor on the visual display is over or within the predefined area of the visual display. In embodiments, the predefined area of the visual display corresponds to the correct answer or solution to the language task.

In embodiments, the language task cannot be completed merely by finger movement across the predetermined path.

In embodiments, the language task cannot be completed merely by hand movement across the predetermined path.

In embodiments, linguistic expression is enhanced. In embodiments, the linguistic expression is verbal, written, or gestural.

In embodiments, wherein linguistic comprehension is enhanced. In embodiments, the linguistic comprehension is verbal, written, or gestural.

In embodiments, the non-fluent aphasia is a post-stroke aphasia.

In embodiments, the non-fluent aphasia is a post-traumatic brain injury aphasia.

In embodiments, the non-fluent aphasia is caused by damage (e.g., by stroke or traumatic brain injury) to the left temporal-frontal-parietal regions in the anterior portion of the left cortex. Non-fluent aphasias are characterized by verbal hesitations, word-substitutions (called “paraphasias”), difficulty with verbal initiation, but generally fair to good comprehension, depending upon the level of severity of the aphasia. Aphasia can be mild to severe, with global aphasia being the most severe, impacting all areas of language. In embodiments, the non-fluent aphasia is one of the following: Broca's aphasia: severe, moderate, or mild; transcortical motor aphasia: severe, moderate, or mild; global aphasia: severe; mixed transcortical aphasia: severe. In embodiments, the non-fluent aphasia is accompanied by a motor speech disorders (e.g., apraxia of speech and/or dysarthria); reading and/or writing difficulties (alexia/agraphia); and/or cognitive difficulties (primarily reduced attention/concentration).

In embodiments, the method enhances improvements in ability in naming action verbs synergistically. Examples of action verbs are words such as “jump” or “lift,” whereas non-action verbs include such words as “think”.

In embodiments, the method enhances improvement in word finding and naming synergistically.

In embodiments, the method enhances improvements in verbal grammar and syntax synergistically.

In embodiments, the method enhances recovery from a dysarthria. Dysarthria affects up to 70% of stroke survivors. Dysarthria is a class of motor-speech disorders that occur in stroke as well as brain injury and developmental disorders (such as CP, muscular dystrophy, developmental delays, etc.) It is caused by damage to parts of the brain that control oral-facial muscle movements.

In embodiments, the method enhances recovery from apraxia of speech (AOS). AOS affects approximately 20% of stroke survivors and most-often co-occurs with aphasia. AOS is an abnormality in initiating, coordinating, or sequencing the muscle movements needed to talk. Oral-facial muscles are not directly impacted as with dysarthria; rather, it is a disorder of motor programming and planning.

Because of the rewiring of hand-arm movements to language-based tasks, written language deficits (alexia/dyslexia), often found in patients with aphasia and traumatic brain injury (as well as developmental disorders) can be improved by the method.

In embodiments, the method enhances gestural language improvements. Gesture is often limited in patients with aphasia, because gestures are linguistically-bound. When patients' ability to gesture meaningfully improves, it can lead to improved word-finding. In embodiments, the method enhances improvements in hand-arm strength and upper limb range-of-motion.

The method impacts one or more of the following domains of language: Verbal fluency (naming nouns; naming verbs; verbal initiation; verbal expansion of utterances (words-phrases-sentences, etc.); automatic utterance generation (e.g., days of week, months of year, counting, etc.); Listening comprehension (following directions; word recognition); Reading comprehension (word to picture association; written word, phrase and sentence comprehension; comprehension of yes/no and multiple choice questions); writing (copying words; spelling; written phrase and sentence generation).

In embodiments, the method enhances certain quantifiable hand-arm therapy outcomes synergistically. In embodiments, the method enhances certain hand-arm therapy outcomes synergistically and others concurrently. In embodiments, the method preferentially enhances the hand-arm therapy outcomes improved synergistically as compared to those only improved concurrently. Quantifiable hand-arm therapy outcomes are assessed by, for example, Fugl-Meyer assessment upper extremity (FMA-UE) assessment of sensorimotor function (see, e.g., Fugl-Meyer A R, Jaasko L, Leyman I, Olsson S, Steglind S: The post-stroke hemiplegic patient. A method for evaluation of physical performance. Scand. J. Rehabil. Med. 1975, 7:13-31, the contents of which are hereby incorporated by reference in their entirety), and also in Wolf Motor Function Test™, the contents of which are hereby incorporated by reference in their entirety.

In embodiments, the methods improve one or more of the following quantifiable outcome parameters in speech and language. In embodiments, the one or more of the following quantifiable outcome parameters in speech and language are improved concurrently. In embodiments, the one or more of the following quantifiable outcome parameters in speech and language are improved concurrently but not synergistically. In embodiments, the one or more of the following quantifiable outcome parameters in speech and language are improved synergistically: Western Aphasia Battery-Revised: Spontaneous speech (e.g., as Western Aphasia Battery-Revised), information content (picture description; conversational speech), fluency, grammatical competence, paraphasic errors, auditory verbal comprehension (e.g., yes/no questions; auditory word recognition; following sequential commands), verbal repetition, naming and word finding (object naming; word fluency (e.g., “name as many animals as you can in 1 minute,” etc.); verbal sentence completion; responsive naming), reading and writing, gesture (production and comprehension), visual-spatial processing.

Boston Diagnostic Aphasia Examination: Same as Western Aphasia Battery-Revised list above, but includes more complex verbal grammar and syntax production and comprehension.

### Philadelphia Naming Test: Object Naming.

Concurrent, as opposed to synergistic, linguistic outcomes include improvements in reading comprehension (including visual scanning and tracking), increased functional/social communication, increased oral articulation/intelligibility. Concurrent upper limb outcomes include increased range-of-motion, increased fine motor coordination, increased functional movement (grabbing, lifting, reaching, etc.). Other concurrent outcomes would include increased motivation, enhanced endurance for intensive treatment, reduced depression and anxiety as a result of consistent feedback and small measurable outcomes, increased overall independence, increased cognitive-linguistic skills (short-term verbal recall, complex linguistic attention/concentration, verbal problem solving, calculation).

In embodiments of the systems or methods, a baseline value quantifiable speech-language parameter of the subject is determined prior to initiation of the method. The baseline speech-language parameter value can be used to calibrate the controllable parameters of the system or method by, for example, the user. In embodiments of the systems or methods, a baseline value quantifiable motor skill parameter of the subject is determined prior to initiation of the method. The baseline motor skill parameter value can be used to calibrate the controllable parameters of the system or method by, for example, the user.

In embodiments, the language task(s) is/are speech-language therapy task(s). In embodiments, the language task(s) is/are speech or language-based cognitive task(s).

A task is accomplished once a predetermined end point has been reached.

In embodiments, the computer processor operationally connected to the robotic upper limb device and the computer processor operationally connected to the visual display are the same computer processor. In embodiments, the computer processor operationally connected to the robotic upper limb device and the computer processor operationally connected to the visual display are different computer processors.

In embodiments, the subject's upper limb used to move the moveable member is the contralateral arm contralateral to the hemisphere in which the traumatic brain injury or stroke lesion predominantly exists. In embodiments, the subject's upper limb used to move the moveable member is the injured arm.

The methods and systems can combine speech, language, cognitive and motor therapies for patients with multiple deficits or injuries that can be customized to patients' needs, can track and record progress across domains (cognitive and motor) and can promote both increased intensity and added efficiency within a structured rehabilitation setting.

In embodiments, no transcranial stimulation is applied to the subject during the method.

As used herein, enhancements can be relative to a control amount or value. A control amount or value is decided or obtained, usually beforehand (predetermined), as a normal or standard value. The concept of a control is well-established in the field, and can be determined, in a non-limiting example, empirically from standard or non-afflicted subjects (versus afflicted subjects, including afflicted subjects having different grades of aphasia and/or motor deficits) on an individual or population basis, and/or may be normalized as desired (in non-limiting examples, for volume, mass, age, location, gender) to negate the effect of one or more variables.

In embodiments, a system is provided for therapeutic treatment of a subject having a non-fluent aphasia and/or speech-language developmental motor disorder and/or a motor-speech disorder, the system comprising:

a. a memory device, wherein the memory device is operable to perform the following steps:
i. obtain, from a care provider associated with the subject via the system, at least one motor goal for the subject;
ii. obtain, from the care provider via the system, at least one language goal for the subject;
iii. store the at least at least one motor goal and the at least one language goal;
iv. obtain a plurality of treatments associated with a plurality of goals, wherein the plurality of treatments comprises:
   1. a plurality of motor tasks associated with a plurality of motor goals of the plurality of goals, the plurality of motor tasks comprising:
      a. one or more predefined paths;
      b. one or more predefined starting positions, each of the one or more predefined starting positions being associated with at least one of the one or more predefined paths; and
      c. one or more predefined finishing positions, each of the one or more predefined finishing positions being associated with at least one of the one or more predefined starting positions; and
   2. a plurality of language tasks associated with a plurality of language goals of the plurality of goals, wherein each of the plurality of language tasks is associated with at least one of the plurality of motor tasks such that each of the plurality of language tasks is at least partially accomplished by at least partially completing the at least one of the plurality of motor tasks; and
v. store the plurality of treatments;

b. one or more processor(s) operatively connected to the memory device, wherein the one or more processor(s) is operable to execute machine-readable instructions;

c. a robotic upper limb device comprising at least one movable member,

wherein the robotic upper limb device is operatively connected to the one or more processor(s) and

wherein the at least one movable member is operable to:
i. move along at least two axis's; and
ii. mechanically couple with at least one upper limb of the subject;

d. an electronic device comprising input circuitry operatively connected to the one or more processor(s), wherein the electronic device is operable to:
i. receive one or more inputs from at least one of the following:
   1. the robotic upper limb device
   2. the subject; and
   3. the care provider; and
ii. communicate the one or more inputs from the electronic device to the one or more processor(s); and

e. a display operatively connected to the input circuitry, the robotic upper limb device, and the one or more processor(s), wherein the display is operable to provide one or more graphical user interfaces based on machine-readable instructions;

wherein the one or more processor(s) is operable to perform the following steps:
i. obtain, from the care provider associated with the subject via the input circuitry of the electronic device, one or more goals comprising:
   a. the at least one motor goal for the subject,
   wherein the at least one motor goal is associated with at least one motor task of the plurality of motor tasks; and
   b. the at least one language goal for the subject,
   wherein the at least one language goal is associated with at least one language task of the plurality of language tasks,
wherein the one or more goals are stored in the memory operatively connected to the one or more processor(s);
ii. obtaining and executing first machine-readable instructions to display a first graphical user interface including a first visual display comprising:
   (A) a cursor indicating a relative position of the at least one movable member of the robotic upper limb,
   wherein the cursor is displayed at a predefined starting position at the beginning of the treatment;
   (B) one or more treatments associated with the one or more goals;
   (C) one or more prompts designed to elicit one or more actions by the subject directed towards accomplishing tasks associated with the one or more treatments; and
   (D) one or more indicators associated with the subject's progress of the one or more treatments,
   wherein the one or more indicators are updated in substantially real-time to reflect the subject's progress of the one or more treatments,
   wherein the execution of the first machine-readable instructions causes the display of the system to display the first graphical user interface,
   wherein the first visual display is operationally connected to the one or more processor(s) and the robotic upper limb device such that movement of the at least one movable member of the robotic upper limb device causes the cursor displayed on the display of the system to move in a manner reciprocal to the movement of the at least one movable member,
   wherein the one or more treatments comprise:
   (A) a first motor task of the at least one motor task,
   wherein the first motor task requires movement of the at least one movable member of the robotic upper limb device and the mechanically coupled at least one upper limb of the subject along a first predefined path of the plurality of predefined paths from a first predefined starting position of the plurality of predefined starting positions to a first predefined finishing position of the plurality of predefined finishing positions,
   wherein the first motor task is associated with the at least one motor goal for the subject; and
   (B) a first language task of the plurality of language tasks,
   wherein the first language task is at least partially accomplished by completing the first motor task;
iii. receiving, by the one or more processor(s), first data indicating movement of the at least one movable member,
   wherein the first data is stored in the memory operatively connected to the one or more processor(s);
iv. obtaining and executing second machine-readable instructions, causing the cursor displayed on the display of the system to move reciprocally with the movement indicated by the first data;
v. repeating steps (iii) and (iv) until the one or more processor(s) determine one or more of the following:
   a. the first motor task is completed;
   b. the first language task is completed; and
   c. a predetermined amount of time associated with the treatment has elapsed;
vi. obtaining and executing third machine-readable instructions to display a second graphical user interface including a second visual display comprising second data indicating one or more of the following:
   a. whether the first motor task was completed within the predetermined amount of time;
   b. whether the first language task was completed within the predetermined amount of time;
   c. a list of completed treatments; and
   d. a list of incomplete treatments;
wherein the execution of the third machine-readable instructions causes the display of the system to display the second graphical user interface, and
   wherein the second data is stored in the memory operatively connected to the one or more processor(s).

In embodiments, completing the first language task comprises completion of movement along the first predefined path and subsequent selection, by the subject, of a predefined area of the display corresponding to a correct solution for the first language task via a selection portion of the robotic upper limb device which is activatable by the subject so as to select an area of the display via the cursor displayed by the display of the system. Activation may occur simply by the subject moving their upper arm so as to move the cursor over the predefined area, or can involve “release” or “dropping” of a dragged item on the visual display within the predefined area, or any other suitable activation, such as a “click” of a trigger after the movement along the predetermined path has been achieved.

In embodiments, the selection by the subject of a predefined area of the display cannot be affected by the subject coming into physical contact with the display, nor by moving a touchpad-based cursor or mouse-based cursor which is not operationally connected to the robotic upper limb device.

In embodiments, the predefined area of the display is not the first predefined starting position.

In embodiments, the movement of the moveable member of the robotic upper limb device is adjustable, so as to assist, perturb, constrain or resist motion of the moveable member of the robotic upper limb device by movement of the at least one upper limb of the subject,

wherein the movement of the moveable member of the robotic upper limb device is adjustable by at least one of the following:
(i) a non-subject user and
(ii) fourth machine readable instructions, executed by the one or more processor(s).

In embodiments, the one or more processor(s) are further operable to:

vii. in the event the subject has not completed the first language task within the predetermined amount of time, obtaining and executing fourth machine-readable instructions to display a third graphical user interface including a third visual display comprising one or more prompts designed to elicit one or more actions by the subject directed towards accomplishing the first language task,

wherein the execution of the fourth machine-readable instructions causes the display of the system to display the third graphical user interface.

In embodiments, the one or more processor(s) are further operable to:
vii. in the event the subject has completed the first language task before the predetermined amount of time has elapsed, obtaining and executing fourth machine-readable instructions to display a third graphical user interface including a third visual display comprising a second language task of the plurality of language tasks, wherein the execution of the fourth machine-readable instructions causes the display of the system to display the third graphical user interface.

In embodiments, the one or more processor(s) are further operable to:
vii. in the event a first period of time has elapsed without any performance by the subject, repeating steps (e)(ii) through (e)(iv) iteratively so as to thereby enhance recovery in the subject from a non-fluent aphasia over a period of time or so as to thereby enhance speech-language therapy in a subject with a speech-language developmental motor disorder of the period of time.

In embodiments, the robotic upper limb device is further operable to adjust a resistance to movement of the movable member of the robotic upper limb device.

In embodiments, the one or more processor(s) is further operable to adjust the resistance to movement of the movable member, so as to assist, perturb, constrain, or resist motion of the moveable member of the robotic upper limb device by the at least one upper limb of the subject,

wherein, the one or more processor(s) is operable to adjust the resistance by obtaining and executing fourth machine-readable instructions to adjust the resistance to movement of the movable member, and

wherein the execution of the fourth machine-readable instructions causes the resistance to movement to adjust in accordance with the fourth machine-readable instructions.

In embodiments, the resistance to movement of the movable member is adjusted in between one or more iterations of sets of steps e(iii), e(iv), and e(v).

In embodiments, the adjustment of the resistance is proportional to accuracy of the movement of the moveable member along the first predefined path.

In embodiments, the resistance of the movement is adjusted after a first set of steps e(iii), e(iv), and e(v).

In embodiments, the resistance of the movement is adjusted to assist the subject in movement of the movable member.

In embodiments, the movement of the at least one upper limb of the subject results in completion of the first language task.

In embodiments, the resistance of the movement is adjusted to increase resistance of the movement of the moveable member.

In embodiments, the movement of the at least one upper limb of the subject results in completion of the first language task.

In embodiments, the resistance to movement of the movable member is adjusted by a non-subject user, so as to assist, perturb, constrain, or resist motion of the moveable member of the robotic upper limb device by the at least one upper limb of the subject.

In embodiments, the adjustment of the resistance is proportional to accuracy of the movement of the moveable member along the first predefined path.

In embodiments, the resistance to movement of the movable member is adjusted in between one or more iterations of sets of steps e(iii), e(iv), and e(v).

In embodiments, the adjustment of the resistance is proportional to accuracy of the movement of the moveable member along the first predefined path.

In embodiments, the resistance of the movement is adjusted after a first set of steps e(iii), e(iv), and e(v).

In embodiments, the resistance of the movement is adjusted to assist the subject in movement of the movable member.

In embodiments, the movement of the at least one upper limb, along the predefined path, of the subject is required for completion of the first language task.

In embodiments, the resistance of the movement is adjusted to increase resistance of the movement of the moveable member.

In embodiments, the movement of the at least one upper limb of the subject results in completion of the first language task.

In embodiments, the first language task comprises a second plurality of language tasks, wherein the second plurality of language tasks is a subset of the first plurality of language tasks.

In embodiments, at least one of the second plurality of language tasks is completed by an action comprising completing the first motor task, wherein the first motor task comprises a plurality of individual movements, each of the plurality of individual movements is along a respective predetermined path from a respective starting are to a respective end area.

In embodiments, the first motor task comprises a second plurality of motor tasks, wherein the second plurality of motor tasks is a subset of the first plurality of motor tasks.

In embodiments, the first motor task comprises a second plurality of motor tasks, wherein the second plurality of motor tasks is a subset of the first plurality of motor tasks.

In embodiments, the first language task comprises a verbal/analytical reasoning language task.

In embodiments, the first language task comprises at least one of the following:
(i) a linguistic recall task;
(ii) a phonological task; and
(iii) a speech skill task.

In embodiments, the first language task enhances a connection between word structure and hand-arm movement used in written language.

In embodiments, the first language task engages a pathway used in verbal word finding.

In embodiments, the first language task comprises word identification of a pictured object category.

In embodiments, the word identification of a pictured object category enhances at least one of:
(i) reading comprehension at a word-to-phrase level; and
(ii) word retrieval.

In embodiments, a left frontotemporal brain region of the brain of the subject is simultaneously engaged when accomplishing the first language task and the first motor task.

In embodiments, the system further comprises:
(f) a microphone operatively connected to the one or more processor(s) and operable to:
   (i) receive audio data; and
   (ii) transmit the audio data from the microphone to the one or more processor(s).

In embodiments, the first language task requires:
(i) the subject complete the first motor task; and
(ii) the subject verbalizes one or more words into the microphone at one or more of the following:
   (A) simultaneously with the movement;
   (B) simultaneously with completion of the movement;
   (C) simultaneously with the completion of the first motor task;
   (D) contemporaneously with the movement;
   (E) contemporaneously with completion of the movement; and
   (F) contemporaneously with completion of the first motor task.

In embodiments, the microphone is a head-mounted microphone such that the microphone is affixed to a head of the subject.

In embodiments, the one or more processor(s) is further operable to:
(vii) receive, from the microphone, first audio data representing a response to the first language task;
(viii) generate first text data representing the first audio data by executing speech-to-text functionality on the first audio data;
(ix) analyzing the first text data using natural language understanding to determine whether the first audio data is a correct response to the first language task;
(x) in the case where the one or more processor(s) determine the first audio data is the correct response to the first language task, obtaining and executing fourth machine-readable instructions to display a third graphical user interface including a third visual display comprising a second language task of the plurality of language tasks, wherein the execution of the fourth machine-readable instructions causes the display of the system to display the third graphical user interface; and
(xi) in the case where the one or more processor(s) determine the first audio data is not the correct response to the first language task, obtaining and executing fifth machine-readable instructions to display a fourth graphical user interface including a fourth visual display comprising a message to indicate an incorrect answer and encourage the subject to try again, wherein the execution of the fifth machine-readable instructions causes the display of the system to display the fourth graphical user interface.

In embodiments, the natural language understanding utilizes one or more databases designed to account for one or more subjects recovering from non-fluent aphasia.

In embodiments, the first language task comprises verb naming of an action illustrated on the display of the system.

In embodiments, the first language task comprises noun naming of an object illustrated on the display of the system.

In embodiments, the first language task is completed when:
(i) multiple repetitions of verbalizing one or more words into the microphone are successfully completed; and
(ii) the first motor task is completed.

In embodiments, the first language task enhances the subject's word retrieval.

In embodiments, the memory is further operable to:
(v) obtain a plurality of audio files, each of the audio files corresponding to one or more solutions to one or more language tasks of the plurality of language tasks; and
(vi) store the plurality of audio files, and

wherein the one or more processor(s) is further operable to:
(vii) receive, from the microphone, first audio data representing a response to the first language task;
(viii) obtaining, from the memory, second audio data representing the correct response to the first language task;
(viii) analyzing the first audio data to determine whether the response to the first language task is correct by comparing the first audio data to the second audio data;
(ix) in the case where the one or more processor(s) determine the first audio data is the correct response to the first language task, obtaining and executing fourth machine-readable instructions to display a third graphical user interface including a third visual display comprising a second language task of the plurality of language tasks, wherein the execution of the fourth machine-readable instructions causes the display of the system to display the third graphical user interface; and
(x) in the case where the one or more processor(s) determine the first audio data is not the correct response to the first language task, obtaining and executing fifth machine-readable instructions to display a fourth graphical user interface including a fourth visual display comprising a message to indicate an incorrect answer and encourage the subject to try again, wherein the execution of the fifth machine-readable instructions causes the display of the system to display the fourth graphical user interface.

In embodiments, the first language task comprises a spelling task requiring completion of multiple movements to accomplish the first language task,

wherein the spelling task requires spelling of a first word.

In embodiments, the first word comprises a plurality of letters, and

wherein each letter of the plurality of letters requires movement of the movable member along a different predetermined path within a predefined amount of time.

In embodiments, the first language task is presented in a form of a game, and

wherein gameplay of the game comprises accomplishing the first language task.

In embodiments, the care provider is administrating a language rehabilitative therapy to the subject.

In embodiments, the care provider is administrating motor rehabilitative therapy to the subject.

In embodiments, the care provider targets a speech goal for the subject and adjusts the first language task in accordance with the speech goal for the subject.

In embodiments, the care provider targets a language goal for the subject and adjusts the first language task in accordance with the language goal for the subject.

In embodiments, the care provider targets a speech goal for the subject and adjusts the first motor task in accordance with the speech goal for the subject.

In embodiments, the care provider targets a language goal for the subject and adjusts the first motor task in accordance with the language goal for the subject.

In embodiments, the system is for enhancing recovery from a non-fluent aphasia in the subject.

In embodiments, the non-fluent aphasia is associated with a prior stroke or traumatic brain injury in the subject.

In embodiments, the subject has suffered a prior stroke.

In embodiments, the subject has suffered a prior traumatic brain injury.

In embodiments, the system is for enhancing speech-language therapy in the subject, and wherein the subject has a speech-language developmental motor disorder.

In embodiments, the speech language developmental motor disorder is cerebral palsy.

In embodiments, the speech language developmental motor disorder is associated with one or more of the following:
(i) hemiplegic cerebral palsy;
(ii) Angelman syndrome;
(iii) fragile x syndrome;
(iv) Joubert syndrome;
(v) terminal 22q deletion syndrome;
(vi) Rett syndrome; and
(vii) autism with motor difficulties.

In embodiments, the subject's oral motor control is enhanced by the system.

In embodiments, the robotic upper limb device is an end-effector robotic upper limb device.

In embodiments, the robotic upper limb device is an exoskeleton robotic upper limb device.

The system and method may be personalized, e.g., by the user, to provide language tasks specific to a language goal and/or a motor goal selected. by the user for the subject. The system may prompt or elicit the subject to perform one or more of the specific language tasks (which may involve one or mote of language, speech, spoken and cognitive tasks). In response to a task prompt, the subject may perform the prompted language task.

Based on the completion of the task by the subject subsequent to the prompt, the system may determine whether the user has accomplished or not accomplished the task correctly. if the subject has not correctly accomplished. the task, the system may prompt the subject to perform the task again. in embodiments, if the subject fails to accomplish the task one or more times, the system may prompt the subject to perform a different task. If the subject has correctly accomplished the task, the system may prompt the subject to perform a new task.

In embodiments, based on task accomplishment and/or non-accomplishment by the subject, the system may generate performance data characterizing the subject's performance.

In embodiments, also provided is a programmed product for therapeutic treatment of a subject having a non-fluent aphasia and/or speech-language developmental motor disorder and/or a motor-speech disorder, the system comprising:
1. a memory device, wherein the memory device is operable to perform the following steps:
   1. obtain, from a care provider associated with the subject via the system, at least one motor goal for the subject;
   2. obtain, from the care provider via the system, at least one language goal for the subject;
   3. store the at least at least one motor goal and the at least one language goal;
   4. obtain a plurality of treatments associated with a plurality of goals, wherein the plurality of treatments comprises:
      1. a plurality of motor tasks associated with a plurality of motor goals of the plurality of goals, the plurality of motor tasks comprising:
         1. one or more predefined paths;
         2. one or more predefined starting positions, each of the one or more predefined starting positions being associated with at least one of the one or more predefined paths; and
         3. one or more predefined finishing positions, each of the one or more predefined finishing positions being associated with at least one of the one or more predefined starting positions; and
      2. a plurality of language tasks associated with a plurality of language goals of the plurality of goals, wherein each of the plurality of language tasks is associated with at least one of the plurality of motor tasks such that each of the plurality of language tasks is at least partially accomplished by at least partially completing the at least one of the plurality of motor tasks; and
         5. store the plurality of treatments;
2. one or more processor(s) operatively connected to the memory device, wherein the one or more processor(s) is operable to execute machine-readable instructions;
3. configuration instructions to operate with a robotic upper limb device comprising at least one movable member,
   wherein the robotic upper limb device is operatively connected to the one or more processor(s) and
   wherein the at least one movable member is operable to:
      1. move along at least two axes; and
      2. mechanically couple with at least one upper limb of the subject;
4. an electronic device comprising input circuitry operatively connected to the one or more processor(s), wherein the electronic device is operable to:
   1. receive one or more inputs from at least one of the following:
      1. the robotic upper limb device
      2. the subject; and
      3. the care provider; and
   2. communicate the one or more inputs from the electronic device to the one or more processor(s); and
5. configures instructions to operate a display operatively connected to the input circuitry, the robotic upper limb device, and the one or more processor(s), wherein the display is operable to provide one or more graphical user interfaces based on machine-readable instructions;
   wherein the one or more processor(s) is operable to perform the following steps:
      i. obtain, from the care provider associated with the subject via the input circuitry of the electronic device, one or more goals comprising:
   3. the at least one motor goal for the subject,
      wherein the at least one motor goal is associated with at least one motor task of the plurality of motor tasks; and
   4. the at least one language goal for the subject,
      wherein the at least one language goal is associated with at least one language task of the plurality of language tasks,
      wherein the one or more goals are stored in the memory operatively connected to the one or more processor(s);
      ii. obtaining and executing first machine-readable instructions to display a first graphical user interface including a first visual display comprising:
      (A) a cursor indicating a relative position of the at least one movable member of the robotic upper limb,
      wherein the cursor is displayed at a predefined starting position at the beginning of the treatment;
      (B) one or more treatments associated with the one or more goals;
      (C) one or more prompts designed to elicit one or more actions by the subject directed towards accomplishing tasks associated with the one or more treatments; and
      (D) one or more indicators associated with the subject's progress of the one or more treatments,
      wherein the one or more indicators are updated in substantially real-time to reflect the subject's progress of the one or more treatments,
      wherein the execution of the first machine-readable instructions causes the display of the system to display the first graphical user interface,
      wherein the first visual display is operationally connected to the one or more processor(s) and the robotic upper limb device such that movement of the at least one movable member of the robotic upper limb device causes the cursor displayed on the display of the system to move in a manner reciprocal to the movement of the at least one movable member,
      wherein the one or more treatments comprise:
      (A) a first motor task of the at least one motor task,
      wherein the first motor task requires movement of the at least one movable member of the robotic upper limb device and the mechanically coupled at least one upper limb of the subject along a first predefined path of the plurality of predefined paths from a first predefined starting position of the plurality of predefined starting positions to a first predefined finishing position of the plurality of predefined finishing positions,
      wherein the first motor task is associated with the at least one motor goal for the subject; and
      (B) a first language task of the plurality of language tasks,
      wherein the first language task is at least partially accomplished by completing the first motor task;
      iii. receiving, by the one or more processor(s), first data indicating movement of the at least one movable member,
   wherein the first data is stored in the memory operatively connected to the one or more processor(s);
      iv. obtaining and executing second machine-readable instructions, causing the cursor displayed on the display of the system to move reciprocally with the movement indicated by the first data;
      v. repeating steps (iii) and (iv) until the one or more processor(s) determine one or more of the following:
4. the first motor task is completed;
5. the first language task is completed; and
6. a predetermined amount of time associated with the treatment has elapsed;
   vi. obtaining and executing third machine-readable instructions to display a second graphical user interface including a second visual display comprising second data indicating one or more of the following:
5. whether the first motor task was completed within the predetermined amount of time;
6. whether the first language task was completed within the predetermined amount of time;
7. a list of completed treatments; and
8. a list of incomplete treatments;
   wherein the execution of the third machine-readable instructions causes the display of the system to display the second graphical user interface, and
   wherein the second data is stored in the memory operatively connected to the one or more processor(s).

FIG. 1 is a block diagram of a system for combined rehabilitation for one or more neurological disorders in accordance with exemplary embodiments of the present invention. In embodiments, the system may include the computer system 102, the display 104, and/or the robotic upper limb device 106. In embodiments, the system may further include one or more microphones operatively connected to one or more of the following: the computer system 102, the display 104 and/or the robotic upper limb device 106. The computer system 102, the display 104, microphone, and/or the robotic upper limb device 106, in embodiments, may communicate over network 50. In embodiments, the computer system 102, the display 104, microphone, and/or the robotic upper limb device 106 may communicate with one another locally (e.g., using Bluetooth). The combined rehabilitation, in embodiments, may be administered with the assistance of software being run by the computer system 102. In embodiments, the software being run by the computer system 102 may cause the display 104 to display one or more visual displays associated with the combined rehabilitation. The software, in embodiments, may be operationally connected to the display 104, microphone, and/or the robotic upper limb device 106 such that inputs registered by the display 104 (e.g. a touch screen input), the microphone (e.g., audio data) and/or the robotic upper limb device 106 (e.g. movement of at least a portion of the robotic upper limb device 106) may cause a reciprocal effect with the software which may result in a change in the visual display on the display 104. For example, movement of at least a portion of the robotic upper limb device 106 may cause a cursor being displayed on the display 104 to move in a reciprocal manner on the display 104. In embodiments, the software may include one or more treatments associated with the system 102 and the combined rehabilitation for one or more neurological disorders.

In embodiments, to administer the combined rehabilitation, the robotic upper limb device 106 may be affixed to one or more upper limbs (e.g. hands, arms, wrists, elbows, and/or shoulders of the subject 108, to name a few) of the subject 108. Once the robotic upper limb device 106 is affixed to the subject 108, in embodiments, the computer system 102 may obtain and execute machine learning instructions (e.g. a software program) which may cause the combined rehabilitation to begin. In embodiments, the combined rehabilitation may include one or more of the language tasks and/or motor tasks described below in connection with FIGS. 1B-1 through 1B-5 and FIGS. 3A through 3T, the descriptions of which applying herein.

The computer system 102 may include one or more of the following: one or more processor(s) 102A (hereinafter “processor 102”), memory 102-B, communications circuitry 102-C, one or more microphone(s) 102-D (hereinafter “microphone 102-D”), and/or one or more speaker(s) 102-E (hereinafter “speaker 102-D”), to name a few.

In embodiments, processor 102-A may include any suitable processing circuitry capable of controlling operations and functionality of computer system 102, as well as facilitating communications between various components within computer system 102. In embodiments, processor 102-A may include a central processing unit (“CPU”), a graphic processing unit (“GPU”), one or more microprocessors, a digital signal processor, or any other type of processor, or any combination thereof. In embodiments, the functionality of processor 102-A may be performed by one or more hardware logic components including, but not limited to, field-programmable gate arrays (“FPGA”), application specific integrated circuits (“ASICs”), application-specific standard products (“ASSPs”), system-on-chip systems (“SOCs”), and/or complex programmable logic devices (“CPLDs”). Furthermore, processor 102-A may include its own local memory, which may store program systems, program data, and/or one or more operating systems. However, processor 102-A may run an operating system (“OS”) for computer system 102, and/or one or more firmware applications, media applications, and/or applications resident thereon. In embodiments, processor 102-A may run a local client script for reading and rendering content received from one or more websites. For example, processor 102-A may run a local JavaScript client for rendering HTML or XHTML content received from a particular URL accessed by computer system 102.

Memory 102-B, in embodiments, may store one or more of the following: a plurality of language goals, a plurality of motor goals, a plurality of neurological disorders, a plurality of treatments (e.g. types of treatments, length of treatments, resistance of robotic upper limb device 106 for each treatment, to name a few), subject information (e.g. subject's name, age, medical history, treatment, neurological disorder(s), to name a few), care provider information (e.g. name, age, patients, to name a few), a plurality of language tasks associated with the plurality of language goals, and/or a plurality of motor tasks associated with the plurality of motor goals, to name a few. Memory 102-B, may include one or more types of storage mediums such as any volatile or non-volatile memory, or any removable or non-removable memory implemented in any suitable manner to store data for computer system 102. For example, information may be stored using computer-readable instructions, data structures, and/or program systems. Various types of storage/memory may include, but are not limited to, hard drives, solid state drives, flash memory, permanent memory (e.g., ROM), electronically erasable programmable read-only memory (“EEPROM”), CD ROM, digital versatile disk (“DVD”) or other optical storage medium, magnetic cassettes, magnetic tape, magnetic disk storage or other magnetic storage devices, RAID storage systems, or any other storage type, or any combination thereof. Furthermore, memory 102-B may be implemented as computer-readable storage media (“CRSM”), which may be any available physical media accessible by processor 102-A to execute one or more instructions stored within memory 102-B. In embodiments, one or more applications (e.g., the above described software) may be run by processor(s) 102-A and may be stored in memory 102-B.

In embodiments, communications circuitry 102-C, may include any circuitry allowing or enabling one or more components of computer system 102 to communicate with one another, the display 104, the robotic upper limb device 106, one or more microphones and/or with one or more additional devices, servers, and/or systems, to name a few. As an illustrative example, data retrieved from the robotic upper limb device 106 may be transmitted over a network 50, such as the Internet, to computer system 102 using any number of communications protocols. For example, network 50 may be accessed using Transfer Control Protocol and Internet Protocol (“TCP/IP”) (e.g., any of the protocols used in each of the TCP/IP layers), Hypertext Transfer Protocol (“HTTP”), WebRTC, SIP, and wireless application protocol (“WAP”), are some of the various types of protocols that may be used to facilitate communications between computer system 102 and one or more of the following: one or more components of computer system 102, the display 104, the robotic upper limb device 106, one or more microphones and/or with one or more additional devices, servers, and/or systems, to name a few. In embodiments, computer system 102 may communicate via a web browser using HTTP. Various additional communication protocols may be used to facilitate communications between computer system 102 one or more components of computer system 102, the display 104, the robotic upper limb device 106, one or more microphones and/or with one or more additional devices, servers, and/or systems, to name a few, include the following non-exhaustive list, Wi-Fi (e.g., 802.11 protocol), Bluetooth, radio frequency systems (e.g., 900 MHz, 1.4 GHz, and 5.6 GHz communication systems), cellular networks (e.g., GSM, AMPS, GPRS, CDMA, EV-DO, EDGE, 3GSM, DECT, IS 136/TDMA, iDen, LTE or any other suitable cellular network protocol), optical, BitTorrent, FTP, RTP, RTSP, SSH, and/or VOIP.

In embodiments, communications circuitry 102-C may use any communications protocol, such as any of the previously mentioned exemplary communications protocols. In embodiments, computer system 102 may include one or more antennas to facilitate wireless communications with a network using various wireless technologies (e.g., Wi-Fi, Bluetooth, radiofrequency, etc.). In yet another embodiment, computer system 102 may include one or more universal serial bus (“USB”) ports, one or more Ethernet or broadband ports, and/or any other type of hardwire access port so that communications circuitry 102-C allows computer system 102 to communicate over one or more communications networks via network 50.

Microphones are an optional embodiment. Microphone 102-D, in embodiments, may be a transducer and/or any suitable component capable of detecting audio signals. For example, microphone 102-D may include one or more sensors for generating electrical signals and circuitry capable of processing the generated electrical signals. In embodiments, microphone 102-D may include multiple microphones capable of detecting various frequency levels. As an illustrative example, computer system 102 may include multiple microphones (e.g., four, seven, ten, etc.) placed at various positions about the computer system 102 to monitor/capture any audio outputted in the environment the computer system 102 is located. The various microphones 102-D may include some microphones optimized for distant sounds, while some microphones may be optimized for sounds occurring within a close range of the computer system 102. In embodiments, one or more microphone(s) 102-D may serve as input devices to receive audio inputs, such as speech from the subject 108.

In embodiments, speaker 102-E may correspond to any suitable mechanism for outputting audio signals. For example, speaker 102-E may include one or more speaker units, transducers, arrays of speakers, and/or arrays of transducers that may be capable of broadcasting audio signals and or audio content to a surrounding area where the computer system 102 and/or the display 104 may be located. In embodiments, speaker 102-E may include headphones or ear buds, which may be wirelessly connected, or hard-wired, to the computer device 102 and/or display 104, that may be capable of broadcasting audio directly to the subject 108.

In embodiments, computer system 102 may be hard-wired, or wirelessly connected, to one or more speakers 102-E. For example, the computer device 102 may cause the speaker 102-E to output audio thereon. Continuing the example, the computer system 102 may obtain audio to be output by speaker 102-E, and the computer system 102 may send the audio to the speaker 102-E using one or more communications protocols described herein. For instance, the speaker 102-E, display 104, and/or the computer system 102 may communicate with one another using a Bluetooth® connection, or another near-field communications protocol. In embodiments, computer system 102 and/or display 104 may communicate with the speaker 102-E indirectly.

Display 104, in embodiments, may include one or more processor(s), storage/memory, communications circuitry and/or speaker(s), which may be similar to processor 102-A, memory 102-B, communications circuitry 102-C and speakers 102-E, respectively, the descriptions of which applying herein. The display 104 may be a display screen and/or touch screen, which may be any size and/or shape. In embodiments, display 104 may be a component of the computer system 102 and may be located at any portion of the computer system 102. Various types of displays may include, but are not limited to, liquid crystal displays (“LCD”), monochrome displays, color graphics adapter (“CGA”) displays, enhanced graphics adapter (“EGA”) displays, variable graphics array (“VGA”) display, or any other type of display, or any combination thereof, to name a few. It will be appreciated by those having ordinary skill in the art that the display 104 and the computer system 102 may be separate devices in embodiments, or may be combined into a single device in embodiments. In embodiments, the display 104 may be a touch screen, which, in embodiments, may correspond to a display screen including capacitive sensing panels capable of recognizing touch inputs thereon.

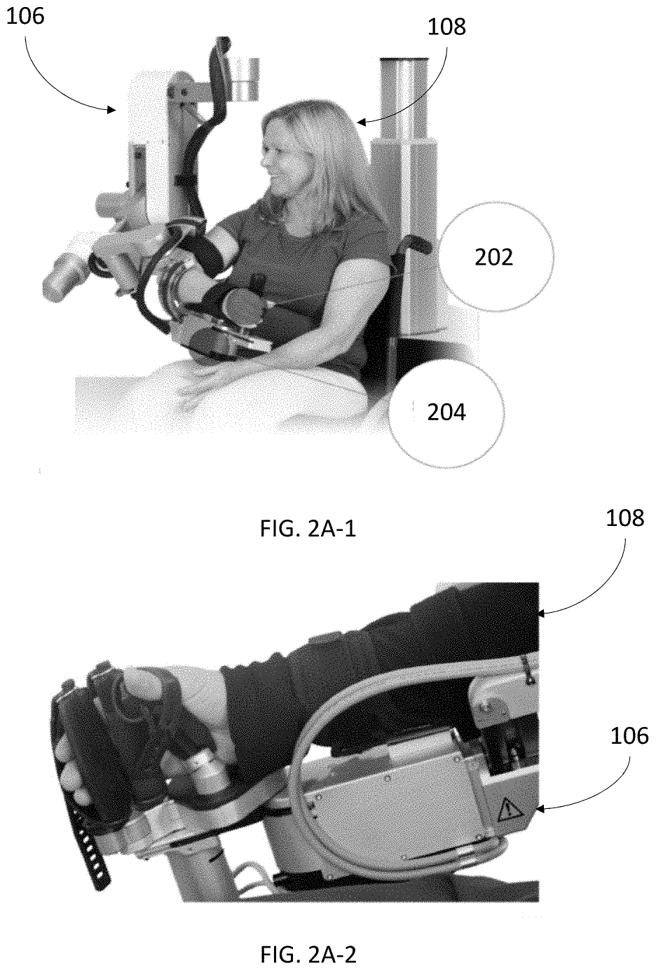

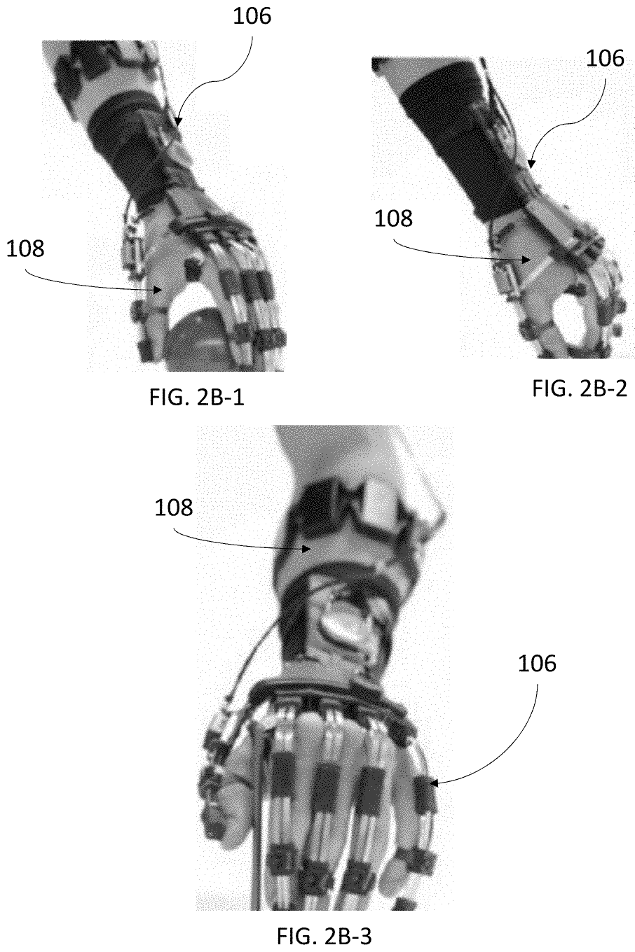

In embodiments, the robotic upper limb device 106 may be an electronic device capable of being affixed to one or more upper limbs of the subject 108. For example, the robotic upper limb device 106 may be an end-effector robotic upper limb device (e.g. the robotic upper limb device 106 described in connection with FIGS. 2A-1 and 2A-2, the descriptions of which applying herein) or an exoskeleton robotic upper limb device (e.g. the robotic upper limb device 106 described in connection with FIGS. 2B-1, 2B-2, and 2B-3, the descriptions of which applying herein). The robotic upper limb device 106, in embodiments, may include one or more processor(s), storage/memory, communications circuitry and/or speaker(s), which may be similar to processor 102-A, memory 102-B, communications circuitry 102-C and speakers 102-E, respectively, the descriptions of which applying herein.

As described above, one or more microphones may be operatively connected to the computer system 102. The one or more microphones may be similar to microphone 102-D, the description of which applying herein.

The computer system 102, in embodiments, as used herein, may correspond to any suitable type of electronic device including, but are not limited to, desktop computers, mobile computers (e.g., laptops, ultrabooks), mobile phones, portable computing devices, such as smart phones, tablets and phablets, televisions, set top boxes, smart televisions, personal display devices, personal digital assistants (“PDAs”), gaming consoles and/or devices, virtual reality devices, smart furniture, and/or smart accessories, to name a few. In embodiments, the computer system 102 may be relatively simple or basic in structure such that no, or a minimal number of, mechanical input option(s) (e.g., keyboard, mouse, track pad) or touch input(s) (e.g., touch screen, buttons) are included. For example, the computer system 102 may be able to receive and output audio, and may include power, processing capabilities, storage/memory capabilities, and communication capabilities. However, in other embodiments, the computer system 102 may include one or more components for receiving mechanical inputs or touch inputs, such as a touch screen and/or one or more buttons.

In embodiments, the computer system 102 may be configured to work with a voice-activated electronic device.

In embodiments, the subject 108 may verbalize one or more words and/or phrases as part of the combined rehabilitation (hereinafter “Response”). The Response, in embodiments, may be detected by the microphone 102-D of the computer system 102 and/or the microphone operatively connected to the computer system 102. The subject 108, for example, may say a Response to a language task associated with the combined rehabilitation. The Response, as used herein, may refer to any question, request, comment, word, words, phrases, and/or instructions that may be spoken to the microphone 102-D of the computer system 102 and/or the microphone operatively connected to the computer system 102.

In embodiments, the microphone 102-D and/or the microphone (hereinafter the “Microphone(s)”) may detect the spoken Response using one or more microphones resident thereon. After detecting the Response, the microphone may send audio data representing Response to the computer system 102. Alternatively, the microphone 102-D may detect the Response and transmit the response to processor 102-A. The microphone 102-D and/or microphone may also send one or more additional pieces of associated data to the computer system 102. Various types of associated data that may be included with the audio data include, but are not limited to, a time and/or date that the Response was detected, an IP address associated with the computer device 102, a type of device, or any other type of associated data, or any combination thereof, to name a few.

The audio data and/or associated data may be transmitted over network 50, such as the Internet, to the computing device 102 using any number of communications protocols. For example, Transfer Control Protocol and Internet Protocol (“TCP/IP”) (e.g., any of the protocols used in each of the TCP/IP layers), Hypertext Transfer Protocol (“HTTP”), and wireless application protocol (“WAP”), are some of the various types of protocols that may be used to facilitate communications between the microphone and the computer system 102.

The computer system 102 may be operatively connected to one or more servers, each in communication with one another, additional microphones, and/or output electronic devices (e.g. display 104), to name a few. Computer system 102, one or more servers, additional microphones, and/or output electronic device may communicate with each other using any of the aforementioned communication protocols. Each server operatively connected to the computer system 102 may be associated with one or more databases or processors that are capable of storing, retrieving, processing, analyzing, and/or generating data to be provided to the computer system 102. For example, each of the one or more servers may correspond to a different type of neurological disorder, enabling natural language understanding to account for different types of speech. The one or more servers, may, in embodiments, correspond to a collection of servers located within a remote facility, and care givers and/or subject 108 may store data on the one or more servers and/or communicate with the one or more servers using one or more of the aforementioned communications protocols.

Referring back to computer system 102, once computer system 102 receives the audio data, computer system 102 may analyze the audio data by, for example, performing speech-to-text (STT) processing on the audio data to determine which words were included spoken Response. Computer system 102 may then apply natural language understanding (NLU) processing in order to determine the meaning of spoken Response. Computer system 102 may further determine whether the Response is correct given the language task being administered by the computer system 102. In embodiments, the correctness of the Response may be determined by comparing the audio data to previously stored audio data (on memory 104) associated with correct answers to the language task being administered by the computer system 102.

In embodiments, whether the Response is correct or not, the computer system 102 may provide an audio and/or visual response to the Response. For example, in embodiments, the response to spoken Response may include content such as, for example, an animation indicating the subject 108 was correct (e.g. a person celebrating a touchdown). Computer system 102 may first determine that output electronic device 300 is associated with voice activated electronic device 10 by looking up the association between voice activated electronic device 10 and output electronic device 300 stored in data structure 102. Upon determining that the content should be output, the computer system 102 may generate first responsive audio data using text-to-speech (TTS) processing. The first responsive audio data may represent a first audio message notifying the subject 108 that the Response was correct (alternatively, not correct). Computer system 102 may play the responsive audio data through speakers 102-E and/or send the responsive audio data to speakers operatively connected to the computer system 102 such that the responsive audio data will play upon receipt.

As noted above, the computer system 102 may also send the content responsive to spoken Response to display 104. For example, in embodiments, computer system 102 may determine that the response to spoken Response should include an animation of a person celebrating. Computer system 102 may retrieve the content (e.g., a gif of a person celebrating) from one or more of the category servers and send the content, along with instructions to display the content, to display 104. Upon receiving the content and instructions, display 104 may display the content

In embodiments, computer system 102 may send instructions to Backend system 100 the display 104 that cause display 104 to output the content and display 104 may obtain the content from a source other than computer system 102. In embodiments, the content may already be stored on the display 104 and thus, computer system 102 does not need to send the content to the display 104. Also, in embodiments, the display 104 may be capable of retrieving content from a cloud-based system other than computer system 102. For example, the display 104 may be connected to a video or audio streaming service other than computer system 102. The computer system 102 may send the display 104 instructions that the display 104 to retrieve and output selected content from the cloud-based system such as the video or audio streaming service.

The computer system may receive input(s) from and/or give instructions or output to the robotic upper arm device wirelessly or in a hard-wired manner. Tracking and/or adjusting (e.g., movement resistance) by the computer of the robotic upper arm device (e.g. the moveable member thereof) can be effected wirelessly or in a hard-wired manner.

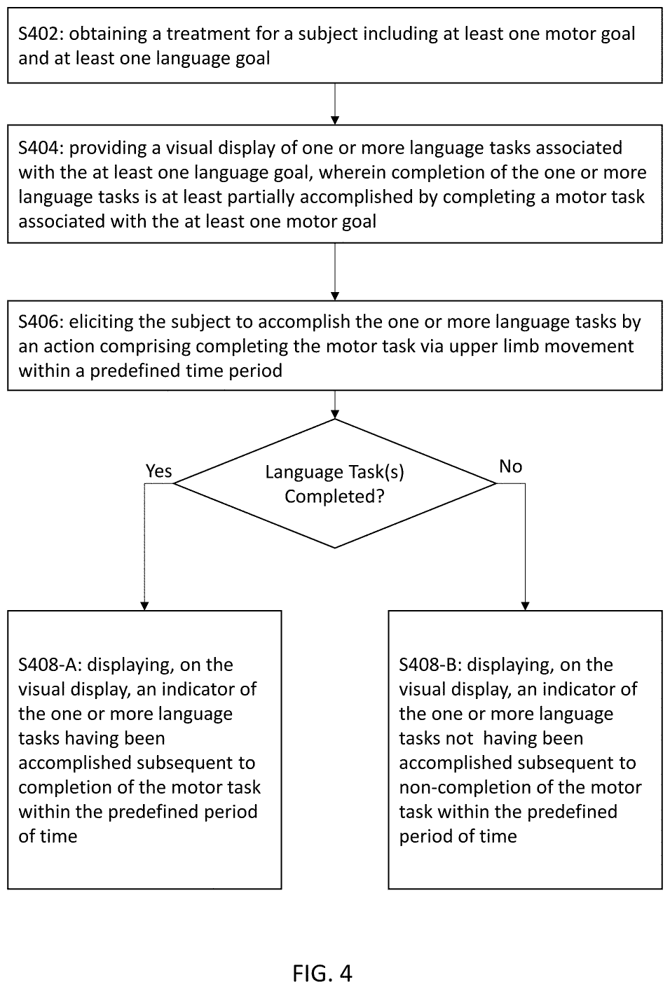

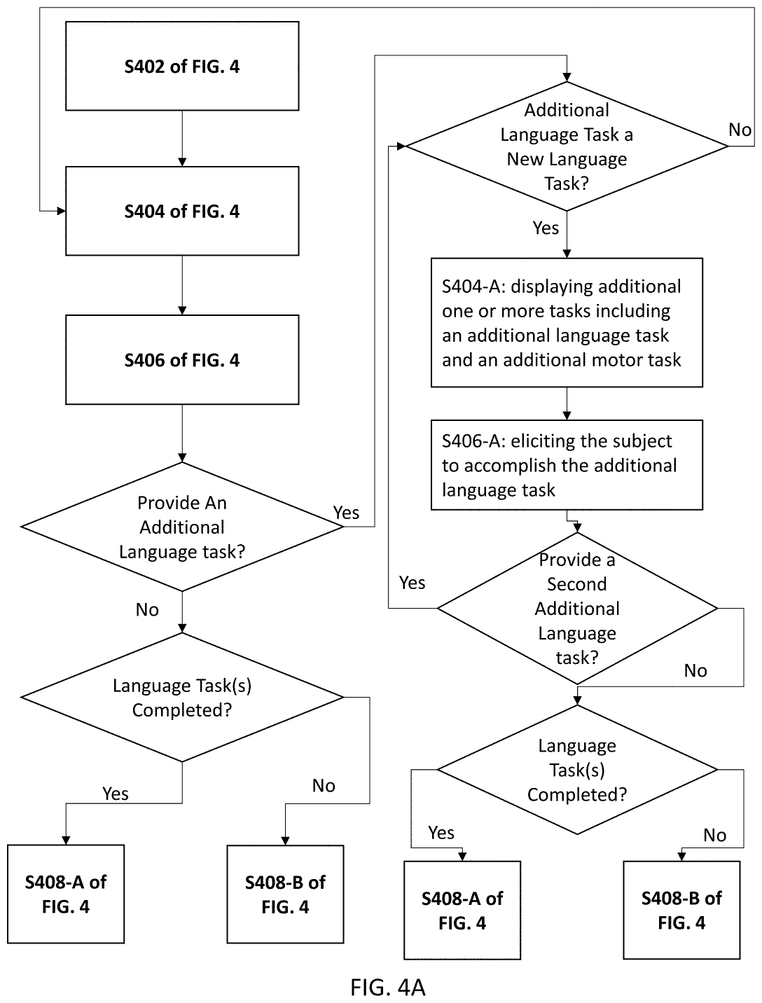

FIG. 4 is a flowchart illustrating an exemplary process for combined rehabilitation for one or more neurological disorders in accordance with exemplary embodiments of the present invention. In embodiments, the combined rehabilitation may be an appropriate treatment for subjects suffering from one or more of the following neurological disorders: non-fluent aphasia, cerebral palsy, hemiplegic cerebral palsy, Angelman syndrome, fragile x syndrome, Joubert syndrome, terminal 22q deletion syndrome, Rett syndrome, and/or autism with motor difficulties, to name a few. In embodiments, non-fluent aphasia may include non-fluent aphasia caused by a stroke and/or a brain injury, to name a few.

In embodiments, the process for combined rehabilitation may begin with step S402. At step S402, in embodiments, a system for combined rehabilitation (hereinafter the “System”) may obtain a treatment for a subject (e.g. subject 108). The treatment, in embodiments, may include at least one motor goal, at least one language goal, and a predetermined amount of time associated with the treatment. The at least one motor goal may be associated with one or more motor tasks. In embodiments, the one or more motor tasks may require movement of the robotic upper limb device along a predefined path from a predefined starting position to a predefined finishing position. The at least one language goal may be associated with one or more language tasks. In embodiments, the one or more language tasks may require the partial completion and/or full completion of one or more motor tasks. The one or more motor tasks and one or more language tasks may be similar to the motor and language tasks described above in connection with FIGS. 1B-1 through 1B-5, the descriptions of which applying herein. The predefined amount of time associated with the treatment may be an amount of time selected by the care provider.

In embodiments, the treatment may be obtained by the System via one or more care providers (e.g. a nurse, physical therapist, doctor, to name a few). In embodiments, the System may obtain information relevant to the subject's treatment, such as one or more of the following: one or more non-fluent aphasia disorders the subject has been diagnosed with, one or more speech-language developmental motor disorders the subject has been diagnosed with, past treatments the subject has accomplished, the resistance of the robotic upper limb device used during past treatments, and/or information regarding the success rate of past treatments, to name a few. In embodiments, the System may include the computer system 102, the display 104, the robotic upper limb device 106, and/or one or more microphones, to name a few.

To begin the treatment, in embodiments, one or more upper limbs of the subject may be affixed to the robotic upper limb device (e.g. robotic upper limb device 106). The process for administering the combined rehabilitation may, in embodiments, continue with step S404. At step S404, in embodiments, the system may provide a visual display of one or more language tasks associated with the at least one language goal. To provide the visual display, in embodiments, the System may obtain and execute first machine-readable instructions. The first machine-readable instructions, in embodiments, may be obtained by accessing local memory and/or by receiving the instructions from an additional computer and/or server. In embodiments, the first machine-readable instructions may be instructions to display a first graphical user interface including the first visual display. The first visual display, in embodiments, may include one or more of the following: a cursor indicating a relative position of a movable member of the robotic upper limb device, the treatment, one or more goals associated with the treatment, one or more prompts designed to elicit one or more actions by the subject directed towards accomplishing tasks (e.g. language tasks, motor tasks, etc.) associated with the treatment, and/or one or more indicators associated with the subject's progress of the treatment, to name a few. In embodiments, upon execution of the first machine-readable instructions, the visual display is displayed by a display of the System. In embodiments, the execution of the first machine-readable instructions causes machine readable instructions to be sent from the computer system 102 of the System to the display 104 of the system, where receipt of such machine-readable instructions causes the display 104 to display the visual display. In embodiments, the visual display may be similar to the displays shown in connection with FIGS. 1B-1 through 1B-5, the descriptions of which applying herein. In embodiments, the System may obtain and execute machine-readable instructions to activate the robotic upper limb device. In embodiments, the machine-readable instructions may include instructions to set the resistance of the robotic upper limb device to a predetermined value. In embodiments, the machine-readable instructions may include instructions to assist the subject with the one or more motor tasks associated with the treatment. In embodiments, execution of the machine-readable instructions results in the activation of the robotic upper limb device.

In embodiments, the process for combined rehabilitation may continue with step S406. At step S406, in embodiments, the System may elicit the subject to accomplish one or more language tasks associated with the treatment by an action via upper limb movement. The action, in embodiments, may include a motor task associated with the treatment. To elicit the action, in embodiments, the System may obtain and execute second machine-readable instructions. The second machine-readable instructions, in embodiments, may be obtained by accessing local memory and/or by receiving the instructions from an additional computer and/or server. In embodiments, the second machine-readable instructions may be instructions to display a second graphical user interface including a second visual display. The second visual display, in embodiments, may include one or more prompts, the amount of time left in the treatment, music, a video, a gif, and/or one or more messages, to name a few.

The subject, in embodiments, may begin treatment. The treatment, in embodiments, may require the subject to move the robotic upper limb device with one or more upper limbs affixed to the robotic upper limb device. Movement of the robotic upper limb device may cause first data to be sent form the robotic upper limb device to one or more processor(s) of the System. The first data, in embodiments, may indicate movement of the robotic upper limb device. Receipt of the first data, in embodiments, may cause the System to obtain and execute third machine-readable instructions. In embodiments, the third machine-readable instructions may be to move the cursor reciprocally with the movement of the robotic upper limb device. In embodiments, the third machine-readable instructions may be to update the progress of the subject's treatment and/or tasks associated with the treatment.

In embodiments, the first data may indicate that the resistance of the robotic upper limb device is too high. In such embodiments, for example, the System may obtain and execute machine-readable instructions to lower the resistance of the robotic upper limb device. The first data, in embodiments, may indicate that the resistance of the robotic upper limb device is too low. In such embodiments, for example, the System may obtain and execute machine-readable instructions to raise the resistance of the robotic upper limb device. In embodiments, the first data may indicate the subject has completed a language task, a motor task, and/or a language and a motor task, to name a few. In such embodiments, the System may obtain and execute machine-readable instructions to display a second motor task and/or language task (the additional tasks may be displayed in a similar manner as described in connection with step S404, the description of which applying herein).

In embodiments, the System may not receive the first data for a predefined amount of time. The lack of data, in embodiments, may indicate one or more of the following: the resistance is too high and/or the subject needs encouragement, to name a few. In such embodiments, the System may obtain and execute machine readable instructions to lower the resistance of the robotic upper limb device and/or to provide visual and/or audio stimulation to elicit the subject to accomplish the one or more tasks associated with the treatment.

In embodiments, steps S404 and S406 may be repeated until one or more of the following occurs: the subject completes the treatment, the predetermined amount of time has elapsed, the one or more motor tasks have been completed, and/or the one or more language tasks have been completed, to name a few. A more detailed description of the iterative repetition of the treatment is located in connection with the description of FIG. 4A, the description of which applying herein.

Referring to FIG. 4A, after step S406, in embodiments, the System may determine whether to provide an additional language task associated with the language goal provided in step S402. In embodiments, if the predefined time limit associated with the language task has elapsed the System may determine to provide an additional language task. In embodiments, if the predefined time limit associated with the language task has elapsed and a predetermined amount of time associated with the treatment has not elapsed, the System may determine to provide an additional language task. If, in embodiments, the System determines to provide an additional language task, the System may determine whether the Additional language task is a new language task. In embodiments, the System may provide the same language task again if one or more of the following is true: the language task provided was completed, but repetition is part of the treatment, the language task provided was not completed, and/or a combination thereof, to name a few. If the System determines to provide the same language task, the process for combined rehabilitation may continue with step S404 of FIG. 4.

In embodiments, the System may determine to provide a new language task associated with the language goal. In such embodiments, the process for administering the combined rehabilitation may continue with step S404-A. At step S404-A, in embodiments, the System may provide a new visual display on the display 104. The new visual display, in embodiments, may include an additional language task and an additional motor task each respectively associated with the aforementioned language goal and motor goal. Providing, the new visual display, in embodiments, may be similar to providing the visual display in step S404, with the exception that the language task and motor task are different than the language and motor tasks provided in step S404. In embodiments, step S404-A may be similar to step S404 described above in connection with FIG. 4, the description of which applying herein.

In embodiments, the process for combined rehabilitation may continue with step S406-A. At step S406-A, in embodiments, the System may elicit the subject to accomplish the additional language task associated with the treatment by an action via upper limb movement. Step S406-A, in embodiments, may be similar to step S406 described above in connection with FIG. 4, the description of which applying herein.

In embodiments, steps S404-A and S406-A may be repeated until one or more of the following occurs: the subject completes the treatment, the predetermined amount of time has elapsed, the one or more motor tasks have been completed, and/or the one or more language tasks have been completed, to name a few. For example, as shown in FIG. 4A, the System may determine whether to provide a second additional language task. The determination may be similar to the above described decision on whether to provide an additional decision, the description of which applying herein. In embodiments, the System may determine to provide a second additional language task, which will result in another iteration of the System determining whether the second additional language task is to be a new language task. If yes, in embodiments, the process may continue with step S404-A. If no, in embodiments, the System may determine whether the language task(s) (e.g. the additional language task and the original one or more language tasks) are completed. If the language task(s) are completed, in embodiments, the process may continue with step S408-A of FIG. 4, which is described below in more detail, the description of which applying herein. If one or more of the language task(s) are not completed, the process may continue with step S408-B of FIG. 4, which is described below in more detail, the description of which applying herein.

Referring back to the System's determination of whether to provide an additional language task, in embodiments, the System may determine to not provide an additional language task. The determination, in embodiments, may be made based on one or more of the following: if one or more of the following is true: the predefined time limit associated with the current language task has not elapsed; and/or the predetermined amount of time associated with the treatment has elapsed, to name a few. Referring back to FIG. 4, in embodiments, if the one or more language tasks have been completed and/or the System has decided to not provide an additional language task, the process for combined rehabilitation may continue with step S408-A. At step S408-A, in embodiments, the System may display an indicator of the one or more language tasks having been accomplished subsequent to the completion of the motor task within the predefined period of time. In embodiments, if the one or more language tasks have not been completed and/or the System has decided to not provide an additional language task, the process for combined rehabilitation may continue with step S408-B. At step S408-B, in embodiments, the System may display an indicator of the one or more language tasks having not been accomplished subsequent to the non-completion of the motor task within the predefined period of time. To display the indicators of steps S408-A and/or S408-B, in embodiments, the System may obtain and execute third machine-readable instructions to display a second visual display including one or more of the following: the aforementioned indicators, the tasks completed, the completed treatments, a history of tasks completed, a history of tasks not completed, a history of treatments completed, and/or a history of treatments not completed, to name a few.

The steps of the processes described in connection with FIGS. 4 and/or 4A may be rearranged and/or omitted.

In embodiments, where any numerical range is provided herein, it is understood that all numerical subsets of that range, and all the individual integers contained therein, are also provided as embodiments of the invention. For example, 1 to 10 includes the subset of 1 to 3, the subset of 5 to 10, etc. as well as every individual integer value, e.g., 1, 2, 3, 4, 5, and so on.

“And/or” as used herein, for example with option A and/or option B, encompasses the separate and separable embodiments of (i) option A; (ii) option B; and (iii) option A plus option B.

All combinations of the various elements described herein are within the scope of the invention unless otherwise indicated herein or otherwise clearly contradicted by context.

This invention and embodiments thereof will be better understood from the Experimental Details, which follow. However, one skilled in the art will readily appreciate that the specific methods and results discussed are merely illustrative of embodiments of the invention as described more fully in the claims that follow thereafter.

The exemplary embodiments of the present invention, as set forth above, are intended to be illustrative, not limiting. The spirit and scope of the present invention is to be construed broadly.

### REFERENCES

Des Roches, C. A., Balachandran, I., Ascenso, E. M., Tripodis, Y., and Kiran, S. (2015). Effectiveness of an impairment-based individualized rehabilitation program using an iPad-based software platform. Front. Hum. Neurosci. 8:1015. doi: 10.3389/fnhum.2014.01015.
Dipietro L, Krebs H I, Volpe B T, et al. Learning, not adaptation, characterizes stroke motor recovery: evidence from kinematic changes induced by robot-assisted therapy in trained and untrained task in the same workspace. IEEE Trans Neural Syst Rehabil Eng. 2012; 20(1):48-57. doi:10.1109/TNSRE.2011.2175008.
Dohle, C. I., Rykman, A., Chang, J. et al. Pilot study of a robotic protocol to treat shoulder subluxation in patients with chronic stroke. J NeuroEngineering Rehabil 10, 88 (2013). https://doi.org/10.1186/1743-0003-10-88.
Godlove, J., Anantha, V., Advani, M., Des Roches, C., Kiran, S. Comparison of therapy practice at home and in the clinic: A retrospective analysis of the Constant Therapy platform data set. Frontiers in Neurology. doi: 10.3389/fneur.2019.00140.
Kiran S, Des Roches C A, Balachandran I, Ascenso E. Development of an impairment-based individualized treatment workflow using an iPad-based software platform. Semin Speech Lang. 2014; 35(1):38-50. doi:10.1055/s-0033-1362995.
Lo, A. C., et al., “Robot Assisted Therapy for Long-Term Upper-Limb Impairment After Stroke,” New England Journal of Medicine, 362(19):1772-83.
Mallet K H, Shamloul R M, Corbett D, et al. RecoverNow: Feasibility of a mobile tablet-based rehabilitation intervention to treat post-stroke communication deficits in the acute care setting. PLoS One. 2016; 11(12):e0167950. doi:10.1371/journal.pone.0167950.
Rodgers H, Bosomworth H, Krebs H I, et al. Robot assisted training for the upper limb after stroke (RATULS): a multicentre randomised controlled trial. Lancet. 2019; 394(10192):51-62. doi:10.1016/S0140-6736(19)31055-4.
The Learning Corp: About Constant Therapy. Online. https://thelearningcorp.com/constant-therapy/.

1-113. (canceled)

114. A method of enhancing recovery from a non-fluent aphasia in a subject comprising:
a) obtaining from a therapy provider at least one language goal and at least one motor goal for a subject;
b) providing the subject with a visual display of one or more language tasks associated with the at least one language goal, wherein the visual display is operationally connected to a computer processor, and which language tasks are accomplished by an action comprising completing a motor task associated with the at least one motor goal, which motor task comprises movement along a predetermined path, from a predefined starting area to a predefined end area, of a moveable member of a robotic upper limb device operationally connected to a computer processor, which moveable member is moved by movement of an upper limb of the subject harnessed in at least a portion of the moveable member, and wherein movement of the moveable member by movement of the upper limb of the subject is translated into corresponding cursor movement on the visual display;
c) eliciting the subject to accomplish the one or more language tasks by an action comprising completing the motor task via upper limb movement which is translated into cursor movement on the visual display, within a predefined time period, wherein movement outside of the predetermined path does not complete the motor task; and
d) displaying on the visual display an indicator of the one or more language tasks having been accomplished subsequent to completion of the motor task within the predefined time period, or displaying on the visual display an indicator of one or more language tasks not having been accomplished subsequent to non-completion of the motor task within the predefined time period.

115. The method of claim 114, wherein accomplishing the one or more language tasks comprises completion of movement along the predetermined path and subsequent selection by the subject of a predefined area of the visual display corresponding to a correct solution for the language task via a selection portion of the robotic upper limb device which is activatable by the subject so as to select an area of the visual display via the cursor on the visual display.

116. The method of claim 114, wherein the selection by the subject of a predefined area of the visual display corresponding to a correct solution for the language task cannot be effected by the subject touching the screen of the visual display, nor by moving a touchpad-based cursor or mouse-based cursor which is not operationally connected to the robotic upper limb device.

117. The method of claim 114, wherein the predefined area of the visual display corresponding to a correct solution for the language task is not the predefined starting area.

118. The method of claim 114, wherein movement of the moveable member of the robotic upper limb device is adjustable by a non-subject user, or by software executed by the computer processor operationally connected thereto, so as to assist, perturb, constrain or resist motion of the moveable member of the robotic upper limb device by the upper limb movement of the subject.

119. The method of claim 114, comprising eliciting the subject who has either failed to accomplish the language task after steps a), b), c) and d) have been performed or who has accomplished the language task after steps a), b), c) and d) have been performed, to accomplish a second or subsequent one or more language tasks by a second or subsequent iteration of steps c) and d).

120. The method of claim 114, further comprising iteratively repeating a plurality of sets of steps c) and d), with a predetermined time period of non-performance in between each set of steps c) and d), so as to thereby enhance recovery in a subject from a non-fluent aphasia over a period of time or so as to thereby enhance speech-language therapy in a subject with a speech-language developmental motor disorder over a period of time.

121. The method of claim 114, wherein movement resistance of the moveable member of the robotic upper limb device is adjusted or adjustable by software executed by the computer processor operationally connected thereto, so as to assist, perturb, constrain or resist motion of the moveable member of the robotic upper limb device by the upper limb movement of the subject.

122. The method of claim 121, wherein movement resistance of the moveable member of the robotic upper limb device is adjusted in between one or more iterations of sets of steps a), b) and c) or one or more iterations of sets of steps b) and c).

123. A method of enhancing speech-language therapy in a subject with a speech-language developmental motor disorder comprising:
a) obtaining from a therapy provider at least one motor goal and at least one language goal for a subject;
b) providing the subject with a visual display of one or more language tasks associated with the at least one language goal, wherein the visual display is operationally connected to a computer processor, and which language tasks are accomplished by an action comprising completing a motor task associated with the at least one motor goal, which motor task comprises movement along a predetermined path, from a predefined starting area to a predefined end area, of a moveable member of a robotic upper limb device operationally connected to a computer processor, which moveable member is moved by movement of an upper limb of the subject harnessed in at least a portion of the moveable member, and wherein movement of the moveable member by movement of the upper limb of the subject is translated into corresponding cursor movement on the visual display;
c) eliciting the subject to accomplish the one or more language tasks by an action comprising completing the motor task via upper limb movement which is translated into cursor movement on the visual display, within a predefined time period, wherein movement outside of the predetermined path does not complete the motor task; and
d) displaying on the visual display an indicator of the one or more language tasks having been accomplished subsequent to completion of the motor task within the predefined time period, or displaying on the visual display an indicator of one or more language tasks not having been accomplished subsequent to non-completion of the motor task within the predefined time period.

124. A system for therapeutic treatment of a subject having a non-fluent aphasia and/or speech-language developmental motor disorder and/or a motor-speech disorder, the system comprising:
a. a memory device, wherein the memory device is operable to perform the following steps:
i. obtain, from a care provider associated with the subject via the system, at least one motor goal for the subject;
ii. obtain, from the care provider via the system, at least one language goal for the subject;
iii. store the at least at least one motor goal and the at least one language goal;
iv. obtain a plurality of treatments associated with a plurality of goals, wherein the plurality of treatments comprises:
1. a plurality of motor tasks associated with a plurality of motor goals of the plurality of goals, the plurality of motor tasks comprising:
a. one or more predefined paths;
b. one or more predefined starting positions, each of the one or more predefined starting positions being associated with at least one of the one or more predefined paths; and
c. one or more predefined finishing positions, each of the one or more predefined finishing positions being associated with at least one of the one or more predefined starting positions; and
2. a plurality of language tasks associated with a plurality of language goals of the plurality of goals, wherein each of the plurality of language tasks is associated with at least one of the plurality of motor tasks such that each of the plurality of language tasks is at least partially accomplished by at least partially completing the at least one of the plurality of motor tasks; and
v. store the plurality of treatments;
b. one or more processor(s) operatively connected to the memory device, wherein the one or more processor(s) is operable to execute machine-readable instructions;
c. a robotic upper limb device comprising at least one movable member,
wherein the robotic upper limb device is operatively connected to the one or more processor(s) and
wherein the at least one movable member is operable to:
i. move along at least two axes; and
ii. mechanically couple with at least one upper limb of the subject;
d. an electronic device comprising input circuitry operatively connected to the one or more processor(s), wherein the electronic device is operable to:
i. receive one or more inputs from at least one of the following:
1. the robotic upper limb device
2. the subject; and
3. the care provider; and
ii. communicate the one or more inputs from the electronic device to the one or more processor(s); and
e. a display operatively connected to the input circuitry, the robotic upper limb device, and the one or more processor(s), wherein the display is operable to provide one or more graphical user interfaces based on machine-readable instructions;
wherein the one or more processor(s) is operable to perform the following steps:
i. obtain, from the care provider associated with the subject via the input circuitry of the electronic device, one or more goals comprising:
a. the at least one motor goal for the subject,
wherein the at least one motor goal is associated with at least one motor task of the plurality of motor tasks; and
b. the at least one language goal for the subject,
wherein the at least one language goal is associated with at least one language task of the plurality of language tasks,
wherein the one or more goals are stored in the memory operatively connected to the one or more processor(s);
ii. obtaining and executing first machine-readable instructions to display a first graphical user interface including a first visual display comprising:
(A) a cursor indicating a relative position of the at least one movable member of the robotic upper limb,
wherein the cursor is displayed at a predefined starting position at the beginning of the treatment;
(B) one or more treatments associated with the one or more goals;
(C) one or more prompts designed to elicit one or more actions by the subject directed towards accomplishing tasks associated with the one or more treatments; and
(D) one or more indicators associated with the subject's progress of the one or more treatments,
wherein the one or more indicators are updated in substantially real-time to reflect the subject's progress of the one or more treatments,
wherein the execution of the first machine-readable instructions causes the display of the system to display the first graphical user interface,
wherein the first visual display is operationally connected to the one or more processor(s) and the robotic upper limb device such that movement of the at least one movable member of the robotic upper limb device causes the cursor displayed on the display of the system to move in a manner reciprocal to the movement of the at least one movable member,
wherein the one or more treatments comprise:
(A) a first motor task of the at least one motor task,
wherein the first motor task requires movement of the at least one movable member of the robotic upper limb device and the mechanically coupled at least one upper limb of the subject along a first predefined path of the plurality of predefined paths from a first predefined starting position of the plurality of predefined starting positions to a first predefined finishing position of the plurality of predefined finishing positions,
wherein the first motor task is associated with the at least one motor goal for the subject; and
(B) a first language task of the plurality of language tasks,
wherein the first language task is at least partially accomplished by completing the first motor task;
iii. receiving, by the one or more processor(s), first data indicating movement of the at least one movable member,
wherein the first data is stored in the memory operatively connected to the one or more processor(s);
iv. obtaining and executing second machine-readable instructions, causing the cursor displayed on the display of the system to move reciprocally with the movement indicated by the first data;
v. repeating steps (iii) and (iv) until the one or more processor(s) determine one or more of the following:
c. the first motor task is completed;
d. the first language task is completed; and
e. a predetermined amount of time associated with the treatment has elapsed;
vi. obtaining and executing third machine-readable instructions to display a second graphical user interface including a second visual display comprising second data indicating one or more of the following:
a. whether the first motor task was completed within the predetermined amount of time;
b. whether the first language task was completed within the predetermined amount of time;
c. a list of completed treatments; and
d. a list of incomplete treatments;
wherein the execution of the third machine-readable instructions causes the display of the system to display the second graphical user interface, and
wherein the second data is stored in the memory operatively connected to the one or more processor(s).
